(19) United States
(12) Patent Application Publication
(10) Pub. No.: US 2008/0119438 A1
(43) Pub. Date: May 22, 2008

(54) Heparin compositions that inhibit clot associated coagulation factors

(76) Inventors: Jeffrey I. Weitz, Ancaster (CA); Jack Hirsh, Hamilton (CA)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/188,246

(22) Filed: Jul. 25, 2005

Related U.S. Application Data
(63) Continuation of application No. 10/019,325, filed on Feb. 27, 2002, now abandoned.
(60) Provisional application No. 60/141,865, filed on Jun. 30, 1999.
(60) Provisional application No. 60/154,744, filed on Sep. 17, 1999.

(51) Int. Cl. A61K 31/727; A61P 7/02

(52) U.S. Cl. 514/56

(57) ABSTRACT

The present invention provides compositions and methods for the treatment of cardiovascular diseases. More particularly, the present invention relates to modifying thrombus formation by administering an agent which, inter alia, is capable of (1) inactivating fluid-phase thrombin and thrombin which is bound either to fibrin in a clot or to some other surface by catalyzing antithrombin; and (2) inhibiting thrombin generation by catalyzing factor Xa inactivation by antithrombin III (ATIII). The compositions and methods of the present invention are particularly useful for preventing thrombosis in the circuit of cardiac bypass apparatus and in patients undergoing renal dialysis, and for treating patients suffering from or at risk of suffering from thrombus-related cardiovascular conditions, such as unstable angina, acute myocardial infraction (heart attack), cerebrovascular accidents (stroke), pulmonary embolism, deep vein thrombosis, arterial thrombosis, etc.

### FIELD OF THE INVENTION

This invention relates generally to compositions and methods for the treatment of cardiovascular disease. More particularly, the present invention relates to modifying thrombus formation and growth by administering a medium molecular weight heparin (MMWH) composition that, inter alia, is capable of (1) inactivating fluid-phase thrombin as well as thrombin which is bound either to fibrin in a clot or to some other surface by catalyzing antithrombin; and (2) inhibiting thrombin generation by catalyzing factor Xa inactivation by antithrombin III (ATIII). In addition, the present invention provides methods and compositions useful for treating cardiovascular disease.

### BACKGROUND OF THE INVENTION

Heparin acts as an anticoagulant by binding to antithrombin and markedly increasing the rate at which it inhibits activated factor X (factor Xa) and thrombin. The interaction of heparin with antithrombin is mediated by a unique pentasaccharide sequence that is randomly distributed on about one-third of the heparin chains. To catalyze thrombin inhibition by antithrombin, heparin must bind simultaneously to the enzyme and the inhibitor. Provision of this bridging function requires pentasaccharide-containing heparin chains with a minimum molecular weight of 5,400 Daltons. Even heparin chains of this minimum size may be of insufficient length to bridge thrombin to antithrombin if the pentasaccharide is located in the middle of the heparin chain rather than at either end. In contrast, longer pentasaccharide-containing heparin chains are able to provide this bridging function regardless of the location of the pentasaccharide within the heparin chain.

Like heparin, low molecular weight heparin (LMWH) also acts as an anticoagulant by activating antithrombin. However, with a mean molecular weight of about 4,500 to 5,000 Daltons, the majority of the LMWH chains are too short to bridge thrombin to antithrombin. Consequently, the inhibitory activity of LMWH against thrombin is considerably less than that of heparin.

Although heparin is an efficient inhibitor of fluid-phase thrombin, it is limited in its ability to inactivate thrombin bound to fibrin, e.g., clot-bound thrombin. The resistance of fibrin-bound thrombin to inactivation by the heparin-antithrombin complex reflects the fact that heparin bridges thrombin to fibrin to form a ternary fibrin-thrombin-heparin complex. Formation of this ternary complex heightens the affinity of thrombin for fibrin 20-fold (from a Kd of 3 μM to an apparent Kd of 150 nM). By occupying the heparin-binding site on thrombin, the heparin chain that tethers thrombin to fibrin prevents heparin within the heparin-antithrombin complex from bridging antithrombin to the fibrin-bound thrombin. This explains why fibrin-bound thrombin is protected from inactivation by the heparin-antithrombin complex.

Moreover, with a mean molecular weight of 4,500 to 5,000 Daltons, the majority of the chains of LMWH are also too short to bridge thrombin to fibrin. However, because most of the LMWH chains also are too short to bridge thrombin to antithrombin, LMWH is a poor inhibitor of both fluid-phase and fibrin-bound thrombin.

In view of the foregoing, there still remains a need in the art for improved heparin compositions that are useful, for example, for inhibiting thrombogenesis associated with cardiovascular disease. An ideal heparin composition would be one which can pacify the clot by inactivating fibrin-bound thrombin and by blocking thrombin generation, thereby preventing the reactivation of coagulation that occurs once treatment is stopped. More particularly, an ideal heparin composition would be one in which the heparin chains are too short to bridge thrombin to fibrin, but are of a sufficient length to bridge antithrombin to thrombin. The present invention fulfills these and other needs.

### SUMMARY OF THE INVENTION

The present invention provides Medium Molecular Weight Heparin (MMWH) compositions comprising heparin chains that are too short to bridge thrombin to fibrin, but that are of a sufficient length to bridge antithrombin to thrombin. Bridging of thrombin to fibrin is only effected by heparin chains that are larger than 12,000 Daltons. Thus, the minimum molecular weight of heparin needed to provide this bridging function is considerably greater than that needed to bridge antithrombin to thrombin. As such, the MMWH compositions of the present invention were designed to fit within this window. With a molecular weight range of about 6,000 to about 12,000 Daltons, the MMWH compositions of the present invention are comprised of heparin chains or sulfated oligosaccharides that are too short to bridge thrombin to fibrin. However, a lower limit of 6,000 Daltons was specifically chosen to ensure that all of the heparin chains of the MMWH compositions are of a sufficient length to bridge antithrombin to thrombin regardless of where the pentasaccharide sequence is located within the heparin chains. For these reasons, the MMWH compositions of the present invention, unlike heparin, inhibit fibrin-bound thrombin and fluid-phase thrombin equally well.

The MMWH compositions of the present invention can pacify the thrombus (or, interchangeably, clot) by inactivating fibrin-bound thrombin, thereby preventing reactivation of coagulation once treatment is stopped, and can block thrombin generation by inhibiting factor Xa. As such, the present invention provides methods of using the MMWH compositions to treat cardiovascular diseases. As explained above, the MMWH compositions of the present invention are a mixture of sulfated oligosaccharides typically having molecular weights ranging from about 6,000 Daltons to about 12,000 Daltons and, even more preferably, from about 8,000 Daltons to about 10,000 Daltons. In a preferred embodiment, the MMMH compounds of the present invention have a mean molecular weight of about 9,000 Daltons. In one embodiment, at least 31% of the MMWH compositions have a molecular weight greater than or equal to 7,800 Daltons. In another embodiment, at least 25% of the MMWH compositions have a molecular weight greater than or equal to 10,000 Daltons. Such MMWH compositions can readily be prepared from standard or unfractionated heparin.

Moreover, the MMWH compositions of the present invention typically have similar anti-factor Xa and anti-factor IIa activities. In a preferred embodiment, the ratio of anti-factor Xa activity to anti-factor IIa activity ranges from about 2:1 to about 1:1 and, more preferably, from about 1.5:1 to about 1:1. In contrast, LMWHs, for example, have significantly more anti-factor Xa activity than anti-factor IIa activity. In a preferred embodiment, the anti-factor Xa activity of the MMWH compositions of the present invention ranges from about 80 U/mg to about 155 U/mg, preferably 90 U/mg to about 150 U/mg and, more preferably, from about 100 U/mg to about 125 U/mg. In an even more preferred embodiment, the MMWH compositions of the present invention have an anti-factor Xa activity of about 115 U/mg. In a preferred embodiment, the anti-factor IIa activity of the MMWH compositions of the present invention ranges from about 20 U/mg to about 150 U/mg, preferably 40 U/mg to about 100 U/mg and, more preferably, from about 60 U/mg to about 75 U/mg. In an even more preferred embodiment, the MMWH compositions of the present invention have an anti-factor IIa activity of about 65 U/mg.

As described above, the MMWH compositions of the present invention comprise heparin chains that are too short to bridge thrombin to fibrin, but are of a sufficient length to bridge antithrombin to thrombin. Consequently, unlike heparin, the MMWH compositions of the present invention inactivate both fibrin-bound thrombin and free thrombin. Moreover, although most low molecular weight heparin (LMWH) chains are of insufficient length to bridge thrombin to fibrin, they are also too short to bridge antithrombin to thrombin. Consequently, the MMWH compositions of the present invention are considerably better than LMWH at inactivating fibrin-bound thrombin. In addition, although hirudin can inactivate fibrin-bound thrombin, it has no effect on thrombin generation because it is a selective inhibitor of thrombin. Consequently, in contrast to hirudin, the MMWH compositions of the present invention inhibit thrombin generation by catalyzing factor Xa inactivation by antithrombin. Thus, by blocking thrombin generation as well as by inhibiting fibrin-bound thrombin, the MMWH compositions of the present invention overcome the limitations of heparin, LMWH and hirudin, particularly in the setting of acute arterial thrombosis.

Selected MMWH compositions of the invention are also contemplated that are enriched for oligosaccharides having an optimal molecular weight range providing particularly advantageous properties as illustrated herein. These MMWH compositions comprise a mixture of oligosaccharides derived from heparin characterized by one, two, three, four, five, or six, or more of the following characteristics:
(a) having antithrombin- and heparin cofactor II (HCII)-related anticoagulant activity in vitro;
(b) the oligosaccharides are too short to bridge thrombin to fibrin, but are of a sufficient length to bridge antithrombin or HCII to thrombin;
(c) having at least 15%, 20%, 25%, 30%, 35%, or 40% oligosaccharides with at least one or more pentasaccharide sequence;
(d) enriched for oligosaccharides having a molecular weight range from about 6,000 to about 11,000; 7,000 to 10,000; 7,500 to 10,000; 7,800 to 10,000; 7,800 to 9,800; or 7,800 to 9,600; 8,000 to 9,600;
(e) the oligosaccharides have a mean molecular weight of about 7,800 to 10,000, preferably 7,800 to 9,800, more preferably 8,000 to 9,800;
(f) at least 30%, 35%, 40%, 45%, or 50% of the oligosaccharides have a molecular weight greater than or equal to 6000 Daltons, preferably greater than or equal to 8000 Daltons;
(g) a polydispersity of 1.1 to 1.5, preferably 1.2 to 1.4, most preferably 1.3;
(h) having similar anti-factor Xa and anti-factor IIa activities, preferably a ratio of anti-factor Xa activity to anti-factor IIa activity from about 2:1 to about 1:1 and, more preferably, from about 1.5:1 to about 1:1;
(i) an anti-factor Xa activity from about 80 IU/mg to about 155 IU/mg, preferably 90 IU/mg to about 130 IU/mg, more preferably, from about 95 IU/mg to about 120 IU/mg and, most preferably 100-110 IU/mg;
(j) an anti-factor IIa activity from about 20 IU/mg to about 150 IU/mg; preferably 40 IU/mg to about 100 IU/mg, more preferably, from about 80 IU/mg to about 100 IU/mg, most preferably about 90-100 IU/mg.

In accordance with an aspect of the invention a selected MMWH composition of the invention has the characteristics (a), (b), (c) and (d); (a) (b), (c), and (e); (b), (c), (e), and (g); (b), (d), (c), (e), and (h); (b) (c), (d), and (g); (b), (e), (g), (i), and (j); (b), (e), (f), (g), (i) and (j); or (a) through (j).

“Enriched for oligosaccharides” refers to a MMWH composition comprising at least 50%, 55%, 60%, 65%, 70%, 75%, or 80% oligosaccharides within a specified or restricted molecular weight range (e.g. 6,000 to 11,000; 7,000 to 10,000, 7,800 to 10,000; 7,800 to 9,800; or 8,000 to 9,600).

As a result of their ability to (1) inhibit fibrin-bound thrombin as well as fluid-phase thrombin by catalyzing antithrombin, and (2) inhibit thrombin generation by catalyzing factor Xa inactivation by antithrombin, the MMWH compositions of the present invention can be used to treat cardiovascular diseases, including unstable angina, acute myocardial infarction (heart attack), cerebral vascular accidents (stroke), pulmonary embolism, deep vein thrombosis, arterial thrombosis, etc. As such, the present invention provides methods and pharmaceutical compositions for treating such cardiovascular diseases.

In one embodiment, the present invention provides a method of treating a thrombotic condition in a subject, the method comprising administering to the subject a pharmacologically acceptable dose of a MMWH composition of the invention. The composition may comprising a mixture of sulfated oligosaccharides having molecular weights ranging from about 6,000 Daltons to about 12,000 Daltons and, even more preferably, of about 8,000 Daltons to about 10,000 Daltons. In a preferred embodiment, the MMWH composition has a mean molecular weight of about 9,000 Daltons. In anther preferred embodiment, the MMWH composition is a selected MMWH composition having an optimal molecular weight range as described herein. In preferred aspects of this embodiment, the thrombotic condition includes, but is not limited to, venous thrombosis (e.g., deep-vein thrombosis), arterial thrombosis and coronary artery thrombosis. In this embodiment, the MMWH composition inhibits thrombus formation and growth, for example, by inhibiting fibrin-bound thrombin and fluid-phase thrombin, and by inhibiting thrombin generation by catalyzing factor Xa inactivation by antithrombin. Preferably, administration of the compounds is achieved by parenteral administration (e.g., by intravenous, subcutaneous and intramuscular injection).

In another embodiment, the present invention provides a method of preventing the formation of a thrombus in a subject at risk of developing thrombosis, the method comprising administering to the subject a pharmacologically acceptable dose of a MMWH composition of the invention. The composition may comprise a mixture of sulfated oligosaccharides having molecular weights ranging from about 6,000 Daltons to about 12,000 Daltons and, even more preferably, of about 8,000 Daltons to about 10,000 Daltons. In a preferred embodiment, the MMWH composition has a mean molecular weight of about 9,000 Daltons. In another embodiment, the MMWH composition is a selected MMWH composition having an optimal molecular weight range as described herein. In one aspect of this embodiment, the subject is at increased risk of developing a thrombus due to a medical condition which disrupts homeostasis (e.g., coronary artery disease, atherosclerosis, etc.). In another aspect of this embodiment, the subject is at increased risk of developing a thrombus due to a medical procedure (e.g., cardiac surgery (e.g., cardiopulmonary bypass), catheterization (e.g., cardiac catheterization, percutaneous transluminal coronary angioplasty), atherectomy, placement of a prosthetic device (e.g., cardiovascular valve, vascular graft, stent, etc.). In this embodiment, the MMWH compositions can be administered before, during or after the medical procedure. Moreover, administration of the MMWH compositions is preferably achieved by parenteral administration (e.g., by intravenous, subcutaneous and intramuscular injection).

The invention also contemplates the use of a MMWH composition of the invention in the preparation of a medicament for treating a thrombotic condition, or preventing the formation of a thrombus in a subject at risk of developing thrombosis; use of a MMWH composition of the invention in the preparation of a medicament for inhibiting fibrin-bound thrombin and thrombin generation in a subject; use of a MMWH composition of the invention in the preparation of a medicament for treating deep vein thrombosis; and use of a MMWH composition of the invention in the preparation of a medicament for preventing pulmonary embolism in a subject.

Other features, objects and advantages of the invention and its preferred embodiments will become apparent from the detailed description which follows.

### BRIEF DESCRIPTION OF THE DRAWINGS

FIGS. 1A and 1B illustrate the effects of varying heparin concentrations on thrombin (IIa) binding to fibrin (A) and on thrombin's apparent affinity for fibrin (B).

FIG. 2 illustrates the percentage of α-thrombin (α-IIa, γ-thrombin (γ-IIa) or RA-thrombin (RA) that binds to fibrin monomer-sepharose in the absence or presence of heparin.

FIG. 3 illustrates the effect of hirugen (Hg), prothrombin fragment 2 (F2) or antibody against exosite 2 (Wab) on thrombin (IIa) binding to fibrin monomer-sepharose in the absence or presence of 250 nM heparin.

FIG. 4 illustrates the ternary fibrin-thrombin-heparin complex wherein thrombin (IIa) binds to fibrin (Fn) via exosite 1 and heparin (Hp) binds to both Fn and exosite 2 on IIa.

FIG. 5 illustrates the effect of fibrin monomer (Fm) on the rates of thrombin inhibition by antithrombin (▪) or heparin cofactor II () in the presence of 100 nM heparin. Each point represents the mean of at least 2 separate experiments, while the bars represent the SD.

FIGS. 6A and 6B illustrate the inhibitory effects of 4 μM fibrin monomer ( ) on the rates of thrombin inhibition by antithrombin (A) or heparin cofactor II (B) in the absence or presence of heparin at the concentrations indicated. Each point represents the mean of at least 2 experiments, while the bars represent the SD.

FIG. 7 illustrates the interaction of γ-thrombin (γ-IIa). Quick 1 dysthrombin (Q1-IIa) or RA-IIa with fibrin (Fn) in the presence of heparin (Hp). Non-productive ternary complexes are formed because γ-IIa and Q1-IIa have an altered exosite 1, whereas RA-IIa has reduced affinity for Hp.

FIG. 8 illustrates the effect of binary or ternary complex formation on the Km for hydrolysis of N-p-Tosyl-Gly-Pro-Arg-p-nitroanilide by α-thrombin (α-IIa), γ-thrombin (γ-IIa), or RA-thrombin (RA-IIa). Binary complexes include thrombin-fibrin (IIa-Fn), and thrombin-heparin (IIa-Hp), whereas the ternary complex is thrombin-fibrin-heparin (IIa-Fn-Hp). Each bar represents the mean of at least two experiments, while the lines represent the SD.

FIG. 9 illustrates the effect of unfractionated heparin (UFH) and a 6,000 Da heparin fraction (MMWH) on thrombin (IIa) binding to fibrin.

FIG. 10 illustrates the inhibitory effects of 4 μM fibrin monomer on the rate of thrombin inhibition by antithrombin (AT) or heparin cofactor II (HCII) in the presence of heparin or a MMWH composition of the present invention. Each bar represents the mean of at least 2 separate experiments, while the lines represent the SD.

FIG. 11 illustrates the cumulative patency in % of standard heparin (SH), low molecular weight heparin (LMWH), a MMWH composition of the present invention, and hirudin (HIR) in the prevention model study.

FIG. 12 illustrates the effect of standard heparin (SH), low molecular weight heparin (LMWH), a MMWH composition of the present invention, and hirudin (HIR) on cumulative blood loss at 30 minutes.

FIGS. 13A and 13B illustrate the efficacy of LMWH and MMWH composition of the present invention, in the arterial thrombosis model (A), and the effect of LMWH and a MMWH composition of the present invention on blood loss (B).

FIG. 14 shows comparative effects of a MMWH composition of the present invention and LMWH on APTT.

FIG. 15 shows comparative effects of LMWH and a MMWH composition of the present invention on the anti-Xa level.

FIG. 16 is a schematic diagram of the procedure.

FIG. 17 shows a modified Wessler model Clot Weight by percentage following treatment with a MMWH composition of the present invention.

FIG. 18 shows a comparison of LMWH and a MMWH composition of the present invention: Prophylaxis model.

FIG. 19 shows a comparison of LMWH and MMWH composition of the present invention: Prophylaxis model.

FIG. 20 shows a modified Wessler model of clot radioactivity by percentage following treatment with a MMWH composition of the present invention.

FIG. 21 is a comparison of LMWH and a MMWH composition of the present invention: prophylaxis model.

FIG. 22 is a comparison of LMWH and a MMWH composition of the present invention: prophylaxis model.

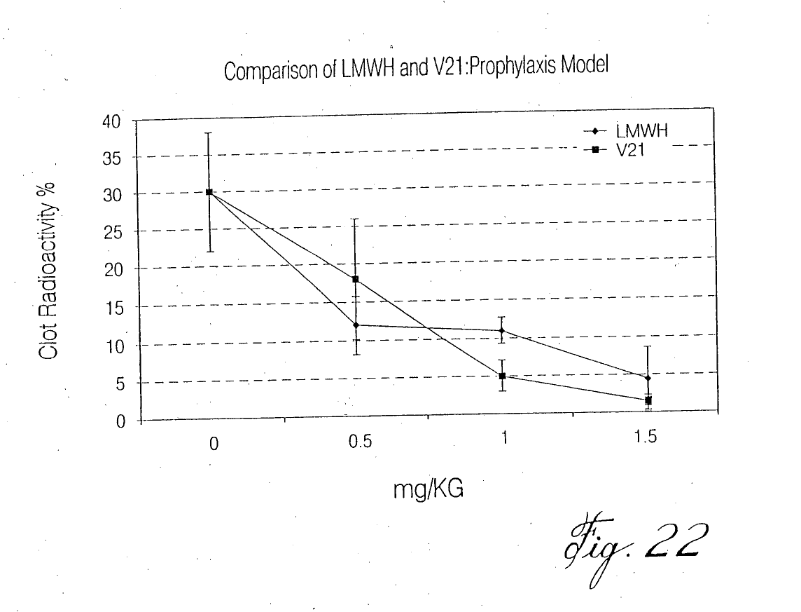

FIG. 23 is a comparison of LMWH and a MMWH composition of the present invention in a treatment model.

FIG. 24 is a comparison of LMWH and a MMWH composition of the present invention in a treatment model.

FIG. 25 shows a comparison of LMWH and a MMWH composition of the present invention on thrombus accretion.

FIG. 26 shows a comparison of LMWH and a MMWH composition of the present invention on thrombus accretion.

FIG. 27 shows treatment of DVT in chronic rabbit model clot accretion with a MMWH composition of the present invention.

FIG. 28 shows treatment of DVT in chronic rabbit model % change in clot weight with a MMWH composition of the present invention.

FIG. 29 is a graph showing rates of AT inhibition of thrombin with heparinase-derived medium molecular weight (MMW) heparins ±4 μM fibrin monomer.

FIG. 30 is a graph showing rates of AT inhibition of thrombin with nitrous acid-derived medium molecular weight (MMW) heparins ±4 μM fibrin monomer.

FIG. 31 is a graph showing rates of AT inhibition of thrombin with periodate-derived medium molecular weight (MMW) heparins ±4 μM fibrin monomer.

FIG. 32 is a graph showing fold inhibition by fibrin monomer of the rate of thrombin inhibition by AT with heparinase and nitrous acid-derived MMW heparins.

FIG. 33 is a graph showing fold inhibition by fibrin monomer of the rate of thrombin inhibition by AT with periodate-derived MMW heparins.

FIG. 34 is a graph showing rates of AT inhibition of Factor Xa with heparinase-derived medium molecular weight heparins.

FIG. 35 is a graph showing rates of AT inhibition of Factor Xa with nitrous acid-derived medium molecular weight heparins.

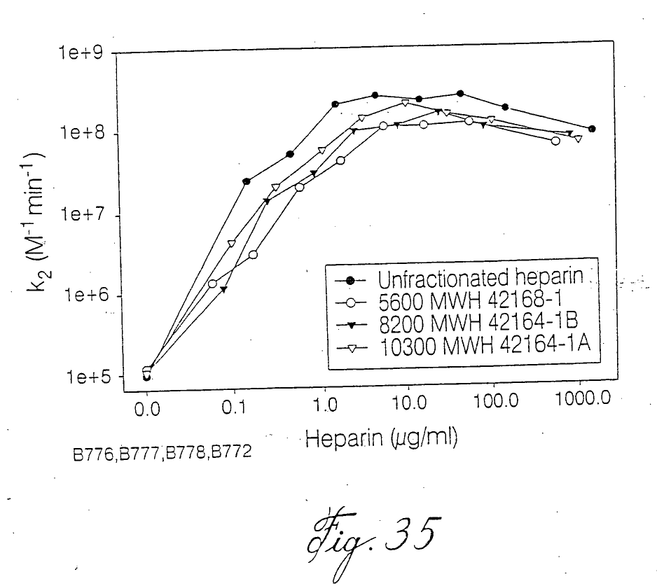

FIG. 36 is a graph showing rates of AT inhibition of Factor Xa with periodate-derived medium molecular weight heparins.

FIG. 37 is a graph showing the effect of UFH and heparinase-derived medium molecular weight heparins on thrombin binding to fibrin clots.

FIG. 38 is a graph showing the effect of UFH and nitrous acid-derived medium molecular weight heparins on thrombin binding to fibrin clots.

FIG. 39 is a graph showing the effect of UFH and periodate-derived medium molecular weight heparins on thrombin binding to fibrin clots.

FIG. 40 is a graph showing the effect of UFH and size restricted heparinase-derived medium molecular weight heparins on thrombin binding to fibrin clots.

FIG. 41 is a graph showing the effect of UFH and size restricted nitrous acid-derived medium molecular weight heparins on thrombin binding to fibrin clots.

### DETAILED DESCRIPTION OF THE INVENTION AND PREFERRED EMBODIMENTS

The present invention provides Medium Molecular Weight Heparin (MMWH) compounds that (1) inhibit fibrin-bound thrombin as well as fluid-phase thrombin by catalyzing antithrombin, and (2) inhibit thrombin generation by catalyzing factor Xa inactivation by antithrombin. These MMWH compositions are a mixture of sulfated oligosaccharides having molecular weights ranging from about 6,000 Daltons to about 12,000 Daltons and, even more preferably, from about 8,000 Daltons to about 10,000 Daltons. In an embodiment, the MMWH compositions of the present invention have a mean molecular weight of about 9,000 Daltons. In one embodiment, at least 31% of the MMWH compositions have a molecular weight greater than or equal to 7,800 Daltons. In another embodiment, at least 25% of the MMWH compositions have a molecular weight greater than or equal to 10,000 Daltons.

More particularly, the MMWH compositions of the present invention can pacify the intense prothrombotic activity of the thrombus. The prothrombotic activity of the thrombus reflects the activity of fibrin-bound thrombin and platelet-bound activated factor X (factor Xa), both of which are relatively resistant to inactivation by heparin and LMWH. This explains why these agents are of limited efficacy in the setting of arterial thrombosis and why rebound activation of coagulation occurs when treatment is stopped. Moreover, although hirudin can, in contrast to heparin, inactivate fibrin-bound thrombin, it fails to block thrombin generation triggered by platelet-bound factor Xa. The ability of hirudin to inactivate fibrin-bound thrombin explains why direct thrombin inhibitors are superior to heparin for the short-term management of arterial thrombosis. However, any beneficial effects of these agents are rapidly lost once treatment is stopped because they fail to block thrombin generation that is triggered by platelet-bound factor Xa.

It has now been determined that fibrin-bound thrombin is resistant to inactivation by heparin because the heparin bridges thrombin to fibrin by binding to both fibrin and the heparin-binding site on thrombin with high affinity; the K_{d} for both the heparin-fibrin and the heparin-thrombin interaction is about 150 nM. Thrombin within this ternary fibrin-thrombin-heparin- complex undergoes a conformational change at its active site that likely limits its reactivity with antithrombin. Furthermore, by occupying the heparin-binding site on thrombin, the heparin chain that tethers thrombin to fibrin prevents heparin within the heparin-antithrombin complex from bridging antithrombin to the fibrin-bound thrombin. This explains why thrombin within the ternary fibrin-thrombin-heparin complex is protected from inactivation by heparin or by LMWH chains that are of sufficient length to bridge thrombin to antithrombin. It is likely that a major contributing factor to both the resistance of acute arterial thrombi to these anticoagulants and rebound activation of coagulation after stopping treatment is the inability of heparin, LMWH or hirudin to pacify the intense prothrombotic activity of the thrombus.

In contrast to heparin, LMWH and hirudin, the MMWH compositions of the present invention can pacify the prothrombotic activity of the thrombus by inactivating fibrin-bound thrombin and by inhibiting thrombin generation by catalyzing factor Xa inactivation by antithrombin. More particularly, it has been discovered that the heparin chains of the MMWH compositions of the present invention are too short to bridge thrombin to fibrin, but are of sufficient length to bridge antithrombin to thrombin. Consequently, unlike heparin, the MMWH compositions of the present invention inactivate both fibrin-bound thrombin and free thrombin. Moreover, although most LMWH chains are of insufficient length to bridge thrombin to fibrin, they are also too short to bridge antithrombin to thrombin. Consequently, the MMWH compositions of the present invention are considerably better than LMWH at inactivating fibrin-bound thrombin. In addition, although hirudin can inactivate fibrin-bound thrombin, it has no effect on thrombin generation because it is a selective inhibitor of thrombin. Consequently, in contrast to hirudin, the MMWH compositions of the present invention inhibit thrombin generation by catalyzing factor Xa inactivation by antithrombin. Thus, by blocking thrombin generation as well as by inhibiting fibrin-bound thrombin, the MMWH compositions of the present invention overcome the limitations of heparin, LMWH and hirudin, particularly in the setting of acute arterial thrombosis.

The MMWH compositions of the present invention typically have similar anti-factor IIa and anti-factor Xa activities. In a presently preferred embodiment, the ratio of anti-factor Xa activity to anti-factor IIa activity ranges from about 2:1 to about 1:1 and, more preferably, from about 1.5:1 to about 1:1. In contrast. LMWHs, for example, have significantly more anti-factor Xa activity than anti-factor IIa activity. In a preferred embodiment, the anti-factor Xa activity of the MMWH compositions of the present invention ranges form about 90 U/mg to about 150 U/mg and, more preferably, from about 100 U/mg to about 125 U/mg. In an even more preferred embodiment, the MMWH compositions of the present invention have an anti-factor Xa activity of about 115 U/mg. In a presently preferred embodiment, the anti-factor IIa activity of the MMWH compositions of the present invention ranges from about 40 U/mg to about 100 U/mg and, more preferably, from about 60 U/mg to about 75 U/mg. In an even more preferred embodiment, the MMWH compositions of the present invention have an anti-factor IIa activity of about 65 U/mg.

Selected MMWH compositions of the invention are also contemplated that are enriched for oligosaccharides having an optimal molecular weight range providing particularly advantageous properties as illustrated herein. These MMWH compositions comprise a mixture of oligosaccharides derived from heparin characterized by having antithrombin- and heparin cofactor II (HCII)-related anticoagulant activity in vitro. The compositions comprise heparin chains that are too short to bridge thrombin to fibrin, but are of a sufficient length to bridge antithrombin or HCII to thrombin. In particular, the compositions have at least 15%, 20%, 25%, 30%, or 40% heparin oligosaccharide chains with at least one or more pentasaccharide sequence. “Pentasaccharide sequence” refers to a key structural unit of heparin that consists of three D-glucosamine and two uronic acid residues (See the structure below). The central D-glucosamine residue contains a unique 3-O-sulfate moiety.

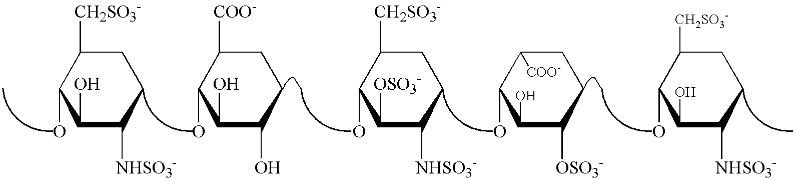

The pentasaccharide sequence represents the minimum structure of heparin that has high affinity for antithrombin (Choay, J. et al., Biochem Biophys Res Comm 1983; 116: 492-499). The binding of heparin to antithrombin through the pentasaccharide sequence results in a conformational change in the reactive center loop which converts antithrombin from a slow to a very rapid inhibitor. Consequently, a selected MMWH composition of the invention will be capable of inhibiting fibrin-bound thrombin as well as fluid-phase thrombin by catalyzing antithrombin, and inhibiting thrombin generation by catalyzing factor Xa inactivation by antithrombin. Preferably, the selected MMWH compositions of the invention are those that inhibit fibrin-bound thrombin and fluid-phase thrombin equally well.

The selected MMWH compositions comprise oligosaccharides having a molecular weight range from about 6,000 to about 11,000. In accordance with one aspect of the invention a MMWH composition is provided that is enriched for oligosaccharides having a molecular weight range of 7,800 to 8,800, preferably 7,800 to 8,600, more preferably 7,800 to 8,500, most preferably 8,000 to 8,500. In another aspect of the invention a MMWH composition is provided that is enriched for oligosaccharides having a molecular weight range of 9,000 to 10,000, preferably 9,200 to 9,800, more preferably 9,300 to 9,600, most preferably 9,400 to 9,600.

In an embodiment the invention also contemplates a MMWH composition of the invention comprising oligosaccharides with a mean molecular weight of 7,800 to 8,800, preferably 7,800 to 8,600, more preferably 7,800 to 8,500, most preferably 8,000 to 8,500. In another embodiment, the invention contemplates a MMWH composition of the invention comprising oligosaccharides with a mean molecular weight of 9,000 to 10,000, preferably 9,200 to 9,800, more preferably 9,300 to 9,600, most preferably 9,400 to 9,600.

A selected MMWH composition may have a polydispersity of 1.1 to 1.5, preferably 1.2 to 1.4, most preferably 1.3.

A selected MMWH composition of the invention may have similar anti-factor Xa and anti-factor IIa activities. In an embodiment, the ratio of anti-factor Xa activity to anti-factor IIa activity ranges from about 2:1 to about 1:1 and, more preferably, from about 1.5:1 to about 1;1. In a preferred embodiment, the anti-factor Xa activity ranges from about 80 IU/mg to about 155 IU/mg, preferably 90 IU/mg to about 130 IU/mg, more preferably, from about 95 IU/mg to about 120 IU/mg and, most preferably 100-110 IU/mg. In a preferred embodiment, the anti-factor IIa activity rages from about 20 IU/mg to about 150 IU/mg; more preferably 40 IU/mg to about 100 IU/mg, and most preferably, from about 80 IU/mg to about 100 IU/mg. In an even more preferred embodiment, the compositions have an anti-factor IIa activity of about 90-100 IU/mg.

The MMWH compositions of the present invention can be prepared from low standard or unfractionated heparin or, alternatively, from low molecular weight heparin (LMWH).

In one embodiment, the MMWH compositions of the present invention can be obtained from unfractionated heparin by first depolymerizing the unfractionated heparin to yield lower molecular weight heparin and then isolating or separating out the MMWH fraction of interest. Unfractionated heparin is a mixture of polysaccharide chains composed of repeating disaccharides made up of a uronic acid residue (D-glucuronic acid or L-iduronic acid) and a D-glucosamine acid residue. Many of these disaccharides are sulfated on the uronic acid residues and/or the glucosamine residue. Generally, unfractionated heparin has an average molecular weight ranging from bout 6,000 Daltons to 40,000 Daltons, depending on the source of the heparin and the methods used to isolate it. the unfractionated heparin used in the process of the present invention can be either a commercial heparin preparation of pharmaceutical quality or a crude heparin preparation, such as is obtained upon extracting active heparin from mammalian tissues or organs. The commercial product (USP heparin) is available from several sources (e.g., SIGMA Chemical Co., St. Louis, Mo.), generally as an alkali metal or alkaline earth salt (most commonly as sodium heparin). Alternatively, the unfractionated heparin can be extracted from mammalian tissues or organs, particularly from intestinal mucosa or lung from, for example, beef, porcine and sheep, using a variety of methods known to those skilled in the art (see, e.g., Coyne, Erwin, Chemistry and Biology of Heparin, (Lundblad, R. L., et al. (Eds.), pp. 9-17, Elsevier/North-Holland, N.Y. (1981)). In a presently preferred embodiment, the unfractionated heparin is porcine intestinal heparin.

Numerous processes for the deploymerization of heparin are known and have been extensively reported in both the scientific and patent literature, and are applicable to the present invention. Such processes are generally based on either chemical or enzymatic reactions. For instance, a lower molecular weight heparin can be prepared from standard, unfractionated heparin by benzylation followed by alkaline depolymerization; nitrous acid deploymerization; enzymatic deploymerization with heparinase; peroxidative depolymerization, etc. Generally methods are chosen that provide compositions with characteristics of a MMWH composition of the invention, in particular a composition of the invention with an optimal molecular weight range. Desired characteristics of a composition of the invention i.e. molecular weight range, mean or average molecular weight, polydispersity, anti-factor Xa activity, anti-factor IIa activity, etc. may be confirmed using standard methods (e.g. see the Examples herein). In a preferred embodiment, a composition of the invention is prepared from unfractionated heparin using nitrous acid depolymerization or heparinase depolymerization.

The unfractionated heparin may be depolymerized by contacting unfractionated heparin, under controlled conditions, to the actions of a chemical agent, more particularly, nitrous acid. The nitrous acid can be added to the heparin directly or, alternatively, it can be formed in situ. To generate the nitrous acid in situ, controlled amounts of an acid are added to a derivative of nitrous acid. Suitable acids include those which advantageously contain biologically acceptable anions, such as acetic acid and, more preferably, hydrochloric acid. Suitable derivatives of nitrous acid include a salt, an ether-salt or, more preferably, an alkali or alkaline-earth salt. In a presently preferred embodiment, a salt of nitrous acid, a water-soluble salt, more preferably, an alkali salt, such as sodium nitrite (NaNO₂), is used.

The depolymerization of unfractionated heparin is preferably carried out in a physiologically acceptable medium, thereby eliminating the problems associated with the use of a solvent that can be detrimental to the contemplated biological applications. Such physiologically acceptable media include, but are not limited to, water and water/alcohol mixtures. In a presently preferred embodiment, water constitutes the preferred reaction medium. In carrying out the depolymerization reaction, it is desirable to use stoichiometric amounts of the reagents (e.g., nitrous acid). The use of stoichiometric amounts of nitrous acid will ensure that when the desired degree of depolymerization is reached, the nitrous acid is entirely consumed. Typically, the weight ratio of unfractionated heparin to sodium nitrite (NaNO₂) ranges from about 100 to 2-4 and, more preferably, from about 100 to 3. Using a stoichiometric amount of nitrous acid avoids the need to “quench” a kinetic (ongoing) reaction with, for example, ammonium sulfamate and, in turn, prevents the formation of mixed salts (e.g., sodium and ammonium) of the lower molecular weight heparin intermediates.

In addition, other parameters, such as temperature and pH., are adjusted with respect to one another in order to obtain the desired products under the most satisfactory experimental conditions. For instance, the depolymerization reaction can be carried out at temperatures ranging from about 0° to 30° C. In fact, temperatures lower then 10° C. can be used for the production of the desired products. However, in a preferred embodiment, the depolymerization reaction is carried out at ambient temperature, i.e., between about 20° C. and 28° C. Moreover, in a preferred embodiment, the depolymerization reaction is initiated and terminated by first lowering and then raising the pH of the reaction mixture. To initiate the depolymerization reaction, the pH of the reaction mixture is lowered to a pH of about 2.5 to 3.5 and, more preferably, to a pH of about 3.0. Similarly, to terminate the depolymerization reaction, the pH of the reaction mixture is raised to a pH of about 6.0 to 7.0 and, more preferably, to a pH of about 6.75. It should be noted that the progress of the reaction can be monitored by checking for the presence or absence of nitrous ions in the reaction mixture using, for example, starch-iodine paper. The absence of nitrous ions in the reaction mixture indicates that the reaction has gone to completion. The time required for the reaction to reach completion will vary depending on the reactants and reaction conditions employed. Typically, however, the reaction will reach completion in any where from about 1 hour to about 3 hours.

Once the reaction has reached completion, the MMWH compositions can be recovered using a number of different techniques known to and used by those of skill in the art. In one embodiment, the MMWH compositions are recovered from the reaction mixture by precipitation, ultrafiltration or chromatography methods. If the desired product is obtained by precipitation, this is generally done using, for example, an alcohol (e.g., absolute ethanol). In a presently preferred embodiment, the MMWH composition is recovered from the reaction mixture using ultrafiltration methods. Ultrafiltration membranes of various molecular weight cuts-offs can advantageously be used to both desalt and define the molecular weight characteristics of the resulting MMWH compositions. Ultrafiltration systems suitable for use in accordance with the present invention are known to and used y those of skill in the art. The commercially available Millipore Pellicon ultrafiltration device is an exemplary ultrafiltration system that can be used in accordance with the present invention. This device can be equipped with various molecular weight cut-off membranes. In a presently preferred embodiment, the resulting MMWH composition is dialyzed or ultrafiltered against purified water (i.e., distilled water (dH₂O)) using a Millipore Pellicon ultrafiltration device equipped with 6,00 Dalton molecular weight cut-off membranes.

After ultrafiltration, the retentate is then lyophilized, i.e., freeze-dried, to give the MMWH composition. The molecular weight characteristics of the resulting MMWH composition can be determined using standard techniques known to and used by those of skill in the art. Such techniques include, for example. GPC-HPLC, viscosity measurements, light scattering, chemical or physical-chemical determination of functional groups created during the depolymerization process, etc. In a preferred embodiment, the molecular weight characteristics of the resulting MMWH composition are determined by high performance size exclusion chromatography in conjunction with multiangle laser light scattering (HPSEC-MALLS). Typically, the resulting MMWH composition has an average or mean molecular weight average (Mw) of about 9,000 Daltons. In a selected composition of the invention, the average or mean molecular weight is about 7,800 to 10,000 Daltons, preferably 7,800 to 9,800 Daltons.

Those of skill in the art will readily appreciate that the resulting MMWH compositions can be subjected to further purification procedures. Such procedures include, but are not limited to, gel permeation chromatography, ultrafiltration, hydrophobic interaction chromatography, affinity chromatography, ion exchange chromatography, etc. Moreover, the molecular weight characteristics of the MMWH compositions of the present invention can be determined using standard techniques known to and used by those of skill in the art as described above. As explained, in a preferred embodiment, the molecular weight characteristics of the MMWH compositions of the present invention are determined by high performance size exclusion chromatography in conjunction with multiangle laser light scattering (HPSEC-MALLS).

MMWH compositions of the invention may be prepared by enzymatic depolymerization of heparin by heparinase (see for example, U.S. Pat. No. 3,766,167, and U.S. Pat. No. 4,396,762). In accordance with one aspect of the invention, a composition of the invention, particularly a selected composition with an optimal molecular weight range or restricted molecular weight range is prepared by a controlled heparinase depolymerization as described in EP0244236 (Nielsen and Ostergard: No. 87303836.8 published Apr. 11, 1987). Using this method a MMWH composition of the invention may be prepared with a desired weight average molecular weight by depolymerizing with heparinase to the corresponding number average molecular weight. The method measures the increase in light absorption (preferably at 230-235 nm i.e. ΔA235) during the course of depolymerization , and depolymerization is stopped when the light absorption has reached a calculated value corresponding to the desired number average molecular weight and the corresponding desired weight average molecular weight.

In another embodiment, the MMWH compositions of the present invention may be obtained by a limited periodate oxidation/hydrolysis of heparin to yield a lower molecular weight heparin, and then isolating or separating out the MMWH fraction of interest. In the first step of this method, heparin is contacted with a limited amount of sodium periodate. In a presently preferred embodiment, the concentration of sodium periodate ranges form about 1 mM to about 50 mM and, more preferably, from about 5 mM to 20 mM. The pH of this reaction mixture ranges form about 3 to 11 and, more preferably, from about 6.5 to about 7.5. The limited periodate oxidation is generally carried out for about 18 hours. In the second step of this method, an alkaline hydrolysis is carried out after the periodate oxidation using metal alkalines, such as NaOH. In a preferred embodiment, the concentration of the metal alkaline, e.g., NaOH, ranges form about 0.1 N to about 1 N and, more preferably, is about 0.25 N. This step is carried out at a temperature ranging from about 0° C. to about 50° C. and, more preferably, at a temperature of about 25° C., for a time period of about 1 hour to about 10 hors and, more preferably, 3 hours. The desired MMWH compositions are obtained using known methods, such as gel-filtration, ion-exchange chromatography, ultrafiltration, dialysis, quaternary ammonium precipitation, and organic solvent precipitation, as described above. Moreover, the MMWH compositions can be further purified using the methods described above.

Using a limited periodate oxidation/hydrolysis method, a MMWH composition is prepared that is structurally distinct from known LMWH compounds. As described above, in one embodiment, the MMWH compositions of the present invention are prepared by a brief treatment of unfractionated heparin with periodate to yield a product that is oxidized at some of the sulfated uronic acid residues. These oxidized sites may be readily cleaved with base. Consequently, cleavage of the MMWH composition may not be random as is typically the case with the methods currently used to prepare LMWH. Moreover, the 2-0 sulfated uronic acid residues that are susceptible to oxidation by periodate are located with some frequency proximal to pentasaccharide sequences. Consequently, the limited periodate/hydrolysis method of the present invention may result in lower molecular weight heparin chains that have the pentasaccharide sequence located at the end of the chain which may leave the remainder of the heparin chain long enough to bridge to thrombin.

The MMWH compositions of the present invention are capable of, inter alia, (1) inhibiting fibrin-bound thrombin as well as fluid-phase thrombin by catalyzing antithrombin, and (2) inhibiting thrombin generation by catalyzing factor Xa inactivation by antithrombin. As such, the MMWH compositions of the present invention can be sued to treat a number of important cardiovascular complications, including unstable angina, acute myocardial infarction (heart attack), cerebral vascular accidents (stroke, pulmonary embolism, deep vein thrombosis, arterial thrombosis, etc. In a preferred embodiment, the MMWH compositions of the present invention are used to treat arterial thrombosis. As such, n another embodiment, the MMWH compositions of the present invention can be incorporated as components in pharmaceutical compositions that are useful for treating such cardiovascular conditions. The pharmaceutical compositions of the present invention are useful either alone or in conjunction with conventional thrombolytic treatments, such as the administration of tissue plasminogen activator (tPA), streptokinase, and the like, with conventional anti-platelet treatments, such as the administration of ticlopidine, and the like, as well as with intravascular intervention, such as angioplasty, atherectomy, and the like.

The MMWH compositions of this invention can be incorporated into a variety of formulations for therapeutic administration. More particularly, the MMWH compositions of the present invention can be formulated into pharmaceutical compositions by combination with appropriate, pharmaceutically acceptable carriers of diluents, and may be formulated into various preparations, preferably in liquid forms, such as slurries, solutions and injections. Administration of the MMWH compositions of the present invention is preferably achieved by parenteral administration (e.g., by intravenous, subcutaneous and intramuscular injection). Moreover, the compounds can be administered in a local rather than systemic manner, for example via injection of the compounds directly into a subcutaneous site, often in a depot or sustained release formulation.

Suitable formulations for use in the present invention are found in Remington's Pharmaceutical Sciences (Mack Publishing Company, Philadelphia, Pa., 17th Ed. (1985)), the teachings of which are incorporated herein by reference. Moreover, for a brief review of methods for drug delivery, see. Langer, Science 249: 1527-1533 (1990), the teachings of which are incorporated herein by reference. The pharmaceutical compositions described herein can be manufactured in a manner that is known to those of skill in the art, i.e., by means of conventional mixing, dissolving, levigating, emulsifying, entrapping or lyophilizing processes. The following methods and excipients are merely exemplary and are in no way limiting.

The MMWH compositions of the present invention are preferably formulated for parenteral administration by injection, e.g., by bolus injection or continuous infusion. Formulations for injection may be presented in unit dosage form, e.g., in ampules or in multi-dose containers, with an added preservative. The compositions may take such forms as suspensions, solutions or emulsions in oily or aqueous vehicles, and may contain formulatory agents such as suspending, stabilizing and/or dispersing agents.

Generally, pharmaceutical formaulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions. Alternatively, the active ingredient may be in powder form for constitution with a suitable vehicle, e.g., sterile pyrongen-free water, before use.

More particularly, for injection, the MMWH compositions can be formulated into preparations by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives, such as solubilizers, isotonic agents, suspending agents, emulsifying agents, stabilizers and preservatives. Preferably, the compositions of the invention may be formulated in aqueous solutions, preferably in physiologically compatible buffers, such as Hanks's solution, Ringer's solution, or physiological saline buffer.

In addition to the formulations described previously, the MMWH compositions can also be formulated as a depot preparation. Such long acting formaulations may be administered by implantation (for example, subcutaneously or intramuscularly) or by intramuscular injection. Thus, for example, the compounds may be formulated with suitable polymeric or hydrophobic materials (for example, as an emulsion in an acceptable oil) or ion exchange resins, or as sparingly soluble derivatives, for example, as a sparingly soluble salt.

Pharmaceutical compositions suitable for use in the present invention include compositions wherein the active ingredients are contained in a therapeutically effective amount. By a “therapeutically effective amount” or, interchangeably, “pharmacologically acceptable dose” or, interchangeably, “anticoagulantly effective amount,” it is meant that a sufficient amount of the compound, i.e., the MMWH composition, will be present in order to achieve a desired result, e.g., inhibition of thrombus accretion when treating a thrombus-related cardiovascular condition, such as those described above by, for example, inactivating clot-bound thrombin, inhibiting thrombin generation by catalyzing factor Xa inactivation by antithrombin, etc. The amount of composition administered will, of course, be dependent on the subject being treated, on the subject's weight, the severity of the affliction, the manner of administration and the judgment of the prescribing physician. Determination of an effective amount is well within the capability of those skilled in the art, especially in light of the detailed disclosure provided herein.

A treatment or composition of the invention may be administered to subjects that are animals, including mammals, and particularly humans. Animals also include domestic animals, including horses, cows, sheep, pigs, cats, dogs, and zoo animals.

Typically, the active product, i.e., the MMWH compositions, will be present in the pharmaceutical composition at a concentration ranging from about 2 μg per dose to 200 μg per dose and, more preferably, at a concentration ranging from about 5 μg per dose to 50 μg per dose. Daily dosages can vary widely. depending on the specific activity of the particular MMWH, but will usually be present at a concentration ranging from about 0.5 μg per kg of body weight per day to about 15 μg per kg of body weight per day and, more preferably, as a concentration ranging from about 1 μg per kg of body weight per day to about 5 μg per kg of body weight per day.

In addition to being useful in pharmaceutical compositions for the treatment of the cardiovascular conditions described above, one of skill in the art will readily appreciate that the active products, i.e., the MMWH compositions, can be used as reagents for elucidating the mechanism of blood coagulation in vitro.

The invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results.

### EXAMPLES

### Example 1

Experimental Findings

### 1.1 Clinical Limitations of Currently Available Anticoagulants

Heparin, LMWH and direct thrombin inhibitors have limitations in acute coronary syndromes. In patients with unstable angina, there is a clustering of recurrent ischemic events after treatment with these agents is stopped (Theroux, P., et al. (1992) N. Engl. J. Med. 327:141-145; Granger, C. B., et al. (1996) Circulation 93:870-888; Oldgren, J., et al. (1996) Circulation 94 (suppl 1):I-431). This is due to reactivation of coagulation because there is an associated elevation in plasma levels of prothrombin fragments F1.2 (F1.2) and fibrinopeptide A (FPA), reflecting increased thrombin generation and thrombin activity, respectively (Granger, C. B., et al. (1995) Circulation 91:1929-1935). In patients with acute myocardial infarction, thrombolytic therapy with tissue plasminogen activator (t-PA) or streptokinase induces a procoagulant state characterized by elevated levels of FPA (Eisenberg, P. R., et al. (1987) J. Am. Coll. Cardiol. 10:527-529; Owen, J., et al. (1988) Blood 72:616-620), which are only partially reduced by heparin (Galvani, J., et al. (1994) J. Am. Coll. Cardiol. 24:1445-1452; Merlini, P. A., et al. (1995) J. Am. Coll. Cardiol. 25:203-209). This explains why adjunctive heparin does not reduce the incidence of recurrent ischemic events in patients receiving streptokinase (Collins, R., et al. (1996) BMJ 313:652-659), and is of only questionable benefit in those given t-PA (Collins, R., et al. (1996) BMJ 313:652-659). Although hirudin is better than heparin both as an adjunct to thrombolytic therapy and in patients with non-Q wave infarction who do not receive thrombolytic agents, the early benefits of hirudin are lost within 30 days (GUSTO Investigators (1996) N. Engl. J., Med. 335(11):775-782). These findings suggest that there is a persistent thrombogenic stimulus that is resistant to both heparin and hirudin.

Similar results are seen in the setting of coronary angioplasty. Recurrent ischemic events occur in 6-8% of patients despite aspirin and high-dose heparin (Popma, J. J., et al (1995) Chest 108:486-501). Although hirudin is superior to heparin for the first 72 hours after successful coronary angioplasty, its benefits are lost by 30 days (Serruys, P. W., et al. (1995) N. Engl. J. Med. 333:757-763). Similarly, at 7 days, hirulog, a semi-synthetic hirudin analogue (Bittl, J. A., et al. (1995) J. Med. 333:764-769), is better than heparin at preventing recurrent ischemic events in patients undergoing angioplasty for unstable angina after acute myocardial infarction; by 30 days, however, there is no difference between hirulog and heparin (Bittl, J. A., et al. (1995) J. Med. 333:764-769). It is likely that both the resistance of acute arterial thrombi to heparin, LMWH and hirudin and the reactivation of coagulation that occurs when treatment is stopped reflect the inability of these anticoagulants to pacify the intense prothrombotic activity of the thrombus.

### 1.2 Factors Responsible for the Prothrombotic Activity of Acute Arterial Thrombi

Arterial thrombosis is triggered by vascular injury. Spontaneous or traumatic rupture of atherosclerotic plaque exposes tissue factor which complexes factor VII/VIIa. The factor VIIa/tissue factor complex then initiates coagulation by activating factors IX and X. Although factor VIIa within the factor VIIa/tissue factor complex is rapidly inactivated by tissue factor pathway inhibitor (Broze G J Jr. (1995) Thromb. Haemost. 74:90-93), arterial thrombi remain thrombogenic.

Studies in vitro have attributed the procoagulant activity of arterial thrombi to (a) thrombin bound to fibrin (Hogg, P. J., et al. (1989) Proc. Natl. Acad. Sci. USA 86:3619-3623; Weitz, J. I., et al., (1990) J. Clin. Invest. 86:385-391), or (b) factor Xa (and possibly factor IXa) bound to platelets within the thrombi (Eisenberg, P. R., et al. (1993) J. Clin. Invest. 91:1877-1883). Fibrin-bound thrombin can locally activate platelets (Kumar, R., et al. (1995) Thromb. Haemost. 74(3):962-968) and accelerate coagulation (Kumar, R., et al. (1994) Thromb. Haemost. 72:713-721), thereby inducing an intense procoagulant state. By triggering thrombin generation , platelet—bound factor Xa (and IXa) augments this procoagulant state.

Both fibrin-bound thrombin and platelet-bound factor Xa are resistant to inactivation by heparin and LMWH (Hogg, P. J., et al. (1989) Proc. Natl. Acad. Sci. USA 86:3619-3623; Weitz, J. I., et al. (1990) J. Clin. Invest. 86:385-391; Teitel, J. M., et al. (1983) J. Clin. Invest. 71:1383-1391; Pieters, J., et al. (1988) J. Biol. Chem. 263:15313-15318), thereby explaining their inability to pacify the procoagulant activity of acute arterial thrombi. Hirudin can inactivate fibrin-bound thrombin (Weitz, J. I., et al. (1990) J. Clin. Invest. 86:385-391), but fails to block thrombin generation triggered by platelet-bound clotting factors. In support of this concept, hirudin reduces the levels of FPA, but has no effect on F1.2 levels in patients with unstable angina (Granger, C. B., et al. (1995) Circulation 91:1929-1935).

There is mounting evidence that both fibrin-bound thrombin and platelet-bound factor Xa contribute to the intense procoagulant activity of thrombi. Thus, the ability of a washed plasma clot to accelerate coagulation when incubated in unanticoagulated whole blood cannot be blocked by either hirudin or tick anticoagulant peptide (TAP), a direct inhibitor of factor Xa that unlike heparin and LMWH inactivates platelet-bound factor Xa as well as free factor Xa (Waxman, L., et al. (1990) Science 248:593-596). In contrast, a combination of hirudin and TAP abolishes the procoagulant activity of plasma clots, suggesting that pacification of acute arterial thrombi requires agents that not only inhibit fibrin-bound thrombin, but also block thrombin generation triggered by platelet-bound factor Xa. Development of these agents requires an understanding of the mechanisms by which fibrin-bound IIa and platelet-bound factor Xa are protected from inactivation by heparin, LMWH and hirudin.

### 1.3 Mechanisms by Which Fibrin-bound Thrombin is Protected from Inactivation by Heparin:

Studies indicate that thrombin binding to fibrin is more complex in the presence of heparin than in its absence, and the consequence of thrombin/fibrin interactions has now been better delineated.

### 1.3.1 Thrombin/Fibrin Interactions in the Absence of Heparin:

In the absence of heparin, α-thrombin binds to fibrin with a Kd=2 μM. Binding is mediated by exosite 1, the substrate-binding site on thrombin (Fenton, J. W. II, et al. (1988) Biochemistry 27:7106-7112) because γ-thrombin (a degraded form of thrombin in which exosite 1 is cleaved) and Quick 1 dysthrombin (a naturally occurring thrombin mutant with Arg 67 within exosite 1 replaced by Cys) fail to bind, whereas RA-thrombin (an exosite 2 mutant (Ye, J., et al. (1994) J. Boil. Chem. 269:17965-17970) with decreased affinity for heparin because Arg residues 93, 97, and 101 are replaced by Ala) binds to fibrin with an affinity similar to that of α-thrombin.

### 1.3.2 Thrombin/Fibrin Interactions in the Presence of Heparin:

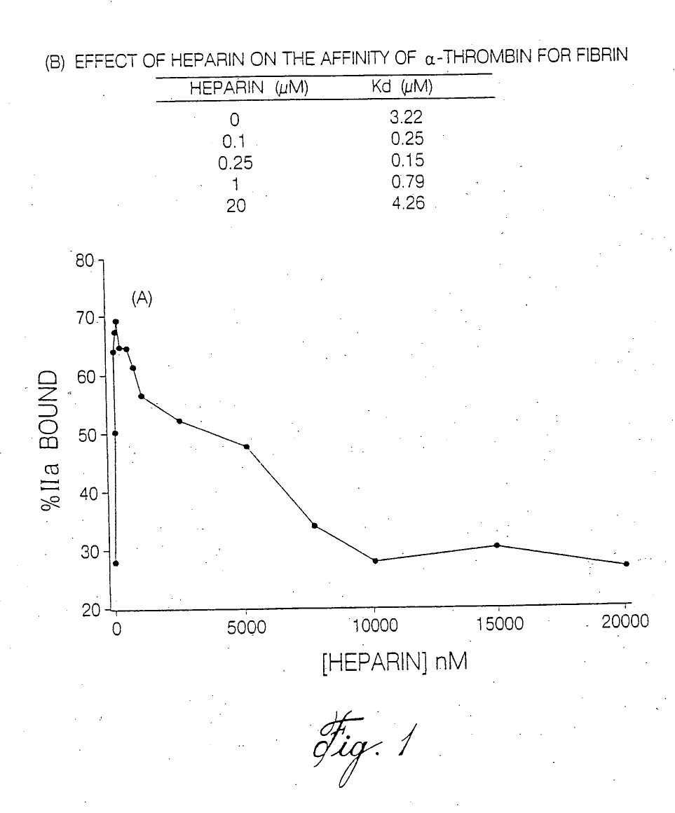

When heparin is present, the amount of thrombin that binds to fibrin changes, as does the mode of thrombin interaction with fibrin. With heparin concentrations up to 250 mM, the amount of thrombin that binds to fibrin increases (FIG. 1A) as does the apparent affinity of thrombin for fibrin (FIG. 1B); at higher heparin concentrations, however, thrombin binding (FIG. 1A) and the affinity of thrombin for fibrin progressively decrease (FIG. 1B). These data extend the results of Hogg and Jackson who demonstrated enhanced thrombin binding to fibrin with fixed concentrations of heparin (see, Hogg, P. J., et al., J. Biol. Chem. 265:241-247 (1990)).

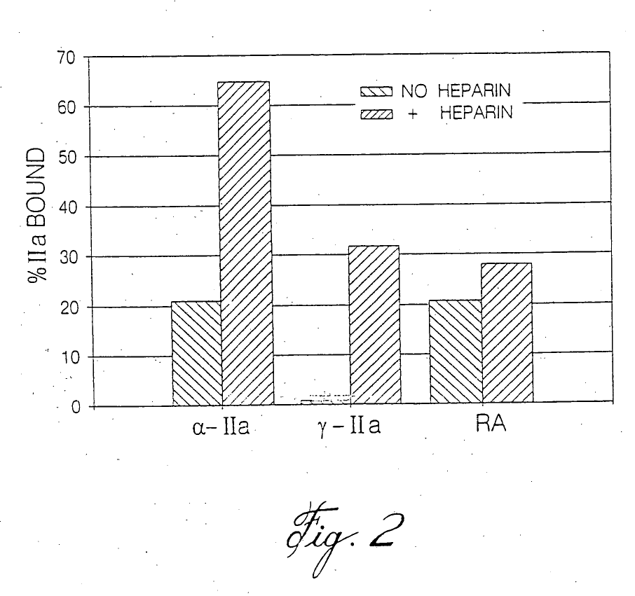

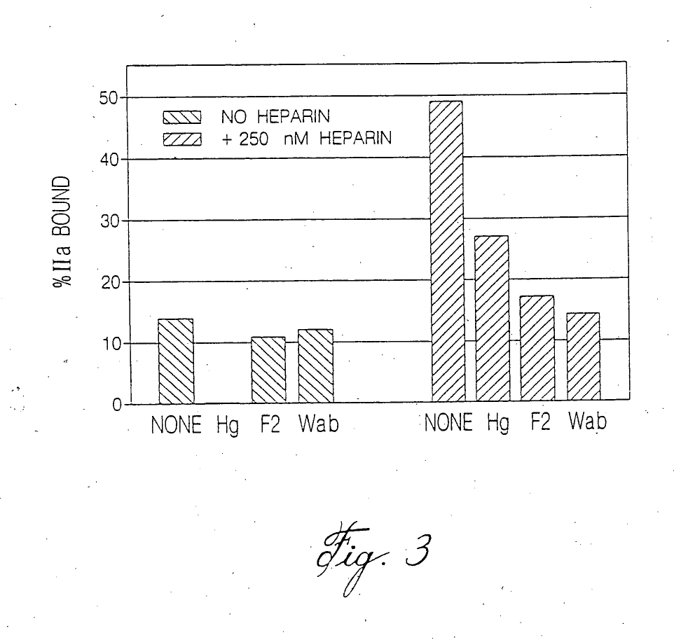

The mode of thrombin binding also changes in the presence of heparin. Whereas thrombin binds to fibrin via exosite 1 in the absence of heparin, enhanced α-thrombin binding seen in the presence of heparin is mediated by exosite 2 because heparin augments the binding of γ-thrombin to the same extent as α-thrombin but has little effect on the binding of TA-thrombin (FIG. 2). Furthermore, excess α-thrombin bound in the presence of heparin is displaced with an antibody to exosite 2 or with prothrombin fragment 2 (F2) which, like heparin, also binds to exosite 2 (Arni, R. K., et al. (1993) Biochemistry 32:4727-4737). In contrast, hirugen, a synthetic analogue of the C-terminal of hirudin (Maraganore, J., et al. (1989) J. Biol. Chem. 264:8692-8698), has no effect on heparin-dependent binding of thrombin (FIG. 3).

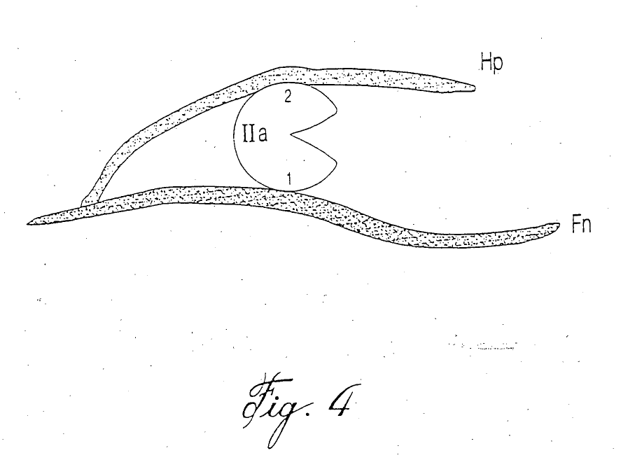

Such findings are interpreted as indicating ternary fibrin-thrombin-heparin complex formation wherein thrombin binds to fibrin directly via exosite 1, and heparin binds to both fibrin and exosite 2 on thrombin (FIG. 4). This occurs because the affinity of heparin for fibrin (Kd=180 nM) is similar to its affinity for α-thrombin (Kd=120 nM). Heparin's interaction with fibrin is pentasaccharide—independent because heparin chains with low affinity for antithrombin bind as tightly as high affinity chains. The biphasic effect of heparin on thrombin binding (FIG. 1) supports the concept of ternary complex formation. Thus, heparin promotes thrombin binding to fibrin until the heparin binding sites are saturated. With higher heparin concentrations, thrombin binding decreases as nonproductive binary fibrin-heparin and thrombin-heparin complexes are formed.

### 1.3.3 Consequences of Thrombin/Fibrin Interactions:

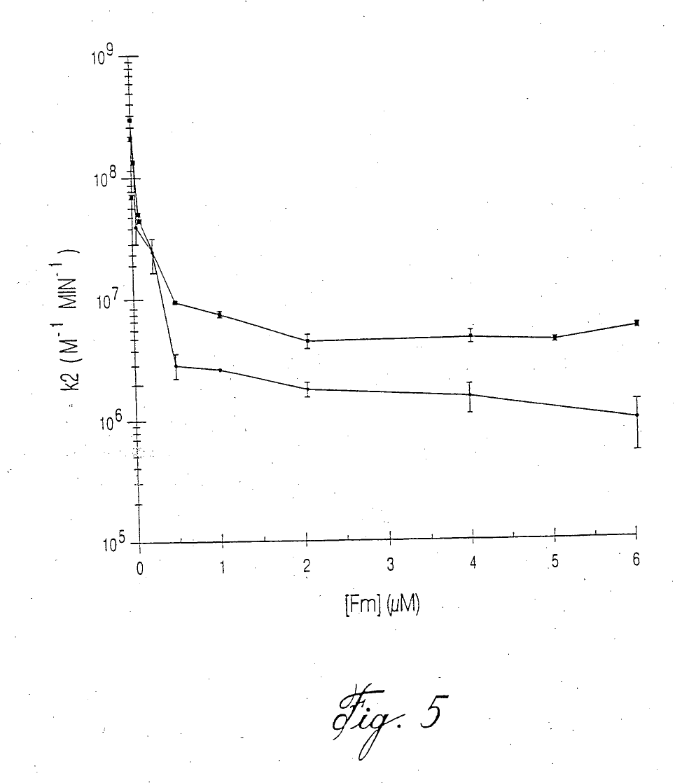

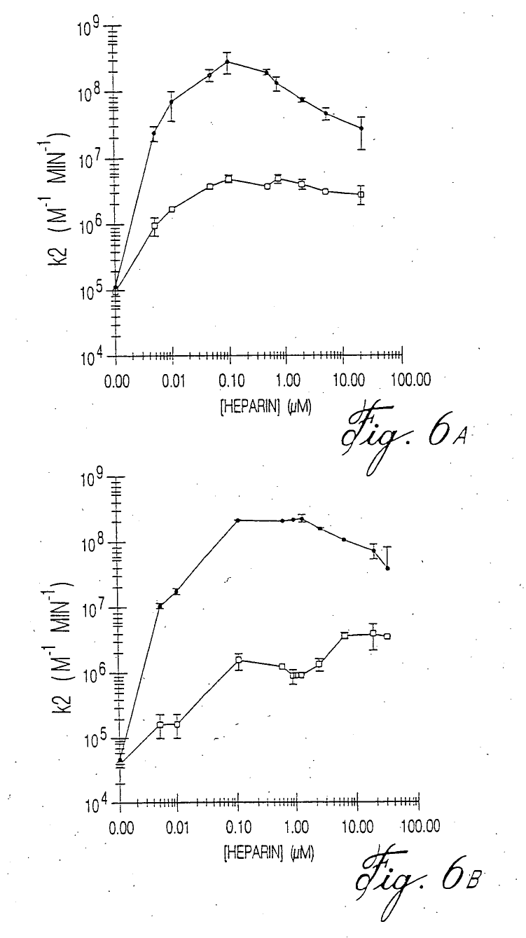

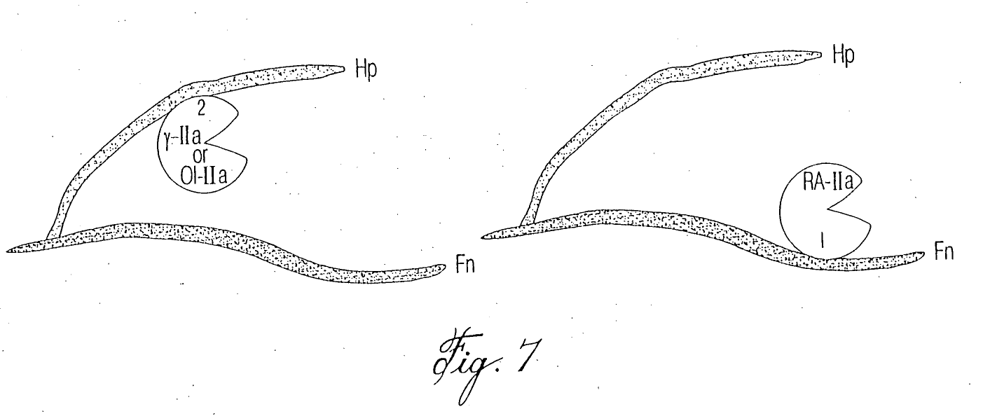

Thrombin within the ternary fibrin—thrombin-heparin complex is protected from inactivation by both antithrombin and heparin cofactor II (HCII). HCII is a naturally occurring antithrombin found in plasma that serves as a secondary inhibitor of thrombin. Thus, the heparin-catalyzed rate of thrombin inactivation by antithrombin or HCII is decreased in the presence of fibrin monomer (FIG. 5). Over a wide range of heparin concentrations, the rates of inactivation by antithrombin and HCII in the presence of saturating amounts of fibrin monomer are up to 60- and 250-fold slower, respectively, than they are in its absence (FIGS. 6A and 6B). For protection to occur, both exosites must be occupied: exosite 1 by fibrin and exosite 2 by heparin. Thus, even though heparin enhances the binding of γ-thrombin and Quick 1 dysthrombin to fibrin by binding to their intact exosite 2 and bridging them to fibrin, neither is protected from inactivation because their altered exosite 1 fails to interact with fibrin (FIG. 7). RA-thrombin is susceptible to inactivation because even though it binds to fibrin with an affinity similar to that of α-thrombin, it has reduced affinity for heparin because of mutations at exosite 2 (FIG. 7).

### 1.3.4 Evidence that Thrombin within the Ternary Fibrin-Thrombin-Heparin Complex Undergoes Allosteric Changes at the Active Site:

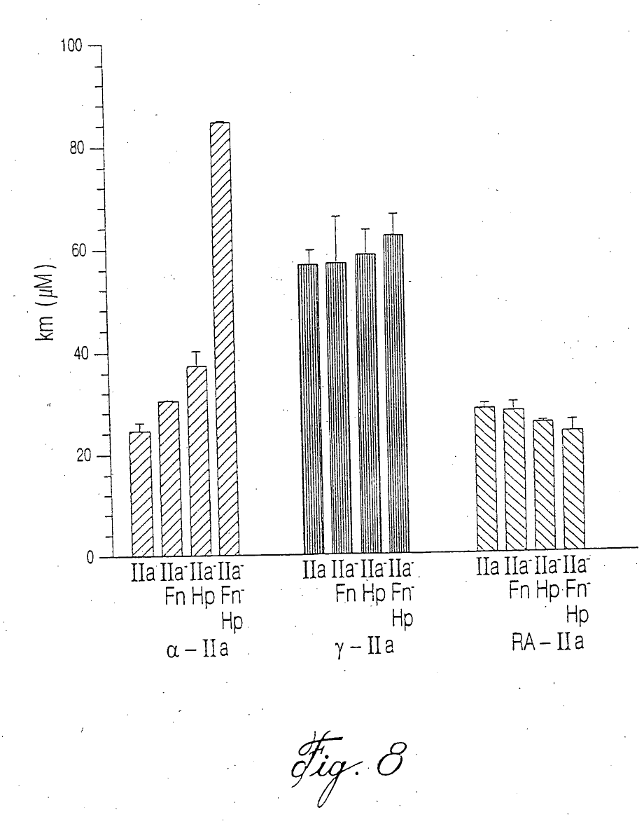

Allosteric changes in the active site of thrombin induced by ternary complex formation likely reduce thrombin reactivity with its substrates or inhibitors. In support of this concept, it has been shown that the rate of thrombin-mediated cleavage of a synthetic substrate is increased when IIa is bound within the ternary fibrin-thrombin-heparin complex, but not with binary thrombin-heparin or thrombin-fibrin complexes (FIG. 8).

### Example 2

### 2.0 Development of Medium Molecular Weight Heparin:

To catalyze thrombin inhibition, heparin bridges antithrombin to thrombin (Daneilsson, A., et al. (1986) J. Biol. Chem. 261:15467-15473). Provision of this bridging function requires heparin chains with a minimal molecular weight of 5,400 (Jordan, R. E., et al. (1980) J. Biol. Chem. 255:10081-10090). Because the majority of LMWH molecules are <5,400 Da, LMWH has little inhibitory activity against thrombin (Jordan, R. E., et al. (1980) J. Biol. Chem. 255:10081-10090). Since heparin bridges thrombin to fibrin to form the ternary fibrin-thrombin-heparin complex, it was hypothesized that this function also requires heparin chains of minimum molecular mass. Further, it was postulated that if this minimum molecular mass is different from that needed to bridge antithrombin to thrombin, there may be a window wherein the heparin chains are too short to bridge thrombin to fibrin, but are of sufficient length to bridge antithrombin to thrombin, thereby overcoming an important mechanism of heparin resistance.

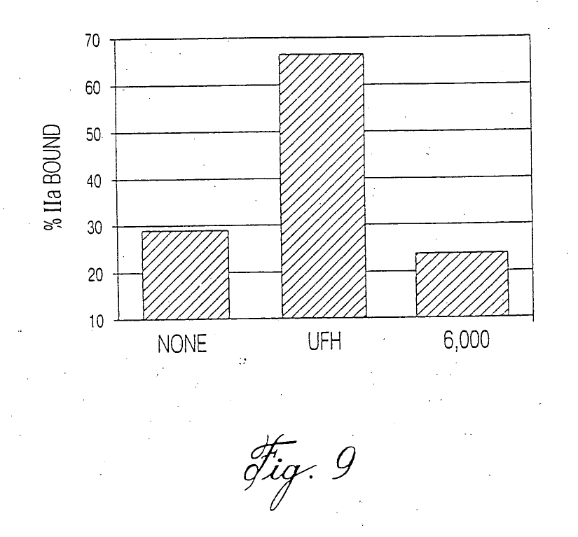

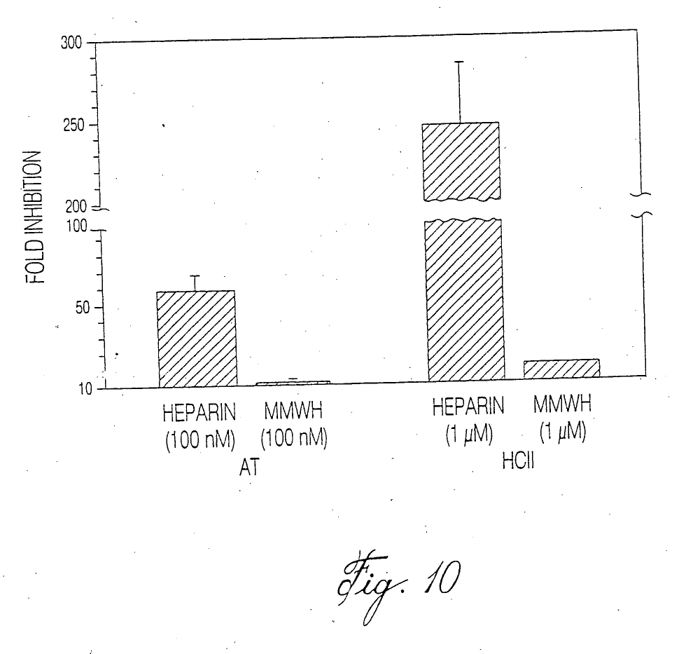

It has now been discovered that such a window exists. For instance, the MMWH compositions of the present invention are long enough to catalyze thrombin inhibition by antithrombin, but do not promote thrombin binding to fibrin (FIG. 9). In contrast to heparin, therefore, the rate of MMWH-catalyzed thrombin inhibition by antithrombin or HCII is almost the same in the presence of fibrin as it is in its absence (FIG. 10).

### 2.1 Characteristics of Medium Molecular Weight Heparin:

Because the chains of MMWH are of sufficient length to bridge antithrombin to thrombin, the anti-factor IIa (i.e., the ability of MMWH to catalyze or activate factor IIa (thrombin) inhibition by antithrombin) is the same as its anti-factor Xa activity (i.e., the ability to catalyze factor Xa inhibition by antithrombin). In contrast, LMWH has greater anti-factor Xa activity than anti-factor IIa activity because more than half of the chains of LMWH are too short to bridge antithrombin to thrombin. Although unfractionated heparin also has equivalent anti-factor Xa and anti-factor IIa activity, it differs from the MMWH compositions of the invention in that it cannot catalyze thrombin inactivation in the presence of fibrin because the chains of unfractionated heparin are long enough to not only bridge antithrombin to thrombin, but also to bridge thrombin to fibrin.

In its typical configuration, the specific activity of the MMWH compositions of the invention is similar to that of unfractionated heparin. Thus, its anti-factor Xa and anti-factor IIa activity may range from 90 to 150 U/mg and 40 to 100 U/mg, respectively, LMWH typically has a specific anti-factor Xa activity of 100 U/mg, whereas its anti-factor IIa activity ranges from 20 to 50 U/mg, depending on the molecular weight profile of the particular LMWH preparation.

### Example 3

### Comparison of the Efficacy and Safety of the MMWH Compositions of the Present Invention with other known Anticoagulants

This example illustrates a study comparing the efficacy and safety of a MMWH composition of the present invention, which is denoted in the figures as V21, LMWH, heparin and hirudin in a the rabbit arterial thrombosis prevention model. The results indicate that the MMWH compositions of the present invention are more effective than LMWH and heparin and safer than hirudin. The arterial thrombosis prevention model was modified so that both efficacy and safety could be assessed in the same animal. Efficacy was assessed by measuring flow over 90 minutes distal to a 95% stenosis in an injured rabbit aorta, and safety was assessed by measuring blood loss over 30 minutes using the rabbit ear model. The four compounds were compared at three dosage levels. Each compound was administered as a bolus and infusion for 90 minutes. The doses listed in the following figures represent the bolus and infusion/60 minutes, administered for 90 minutes. The doses for heparin are shown as units/Kg, for LMWH and V21 as mg/Kg and for hirudin as mg/Kg. V21 has similar anti-Xa activity to LMWH and about twice the anti-IIa activity of LMWH. Thus, the specific activity of LMWH is 100 anti-Xa units/mg and 30 anti-IIa units/mg. The specific activity of V21 is 100 anti-Xa units/mg and 60 anti-IIa units/mg, whereas the specific activity of heparin is about 150 anti-Xa units and 150 anti-IIa units/mg. The anticoagulants were compared in the following dosages. Heparin 50 units/Kg and 75 units/Kg; LMWH and V21 0.5, 1.0 and 1.5 mg/Kg; Hirudin 0.1/0.1, 0.1/0.2 and 0.1/0.3 mg/Kg.

For comparative purposes, 50 units of heparin is equivalent to 0.5 mg of LMWH or V21 in terms of anti-Xa activity, but has more than twice the anti-IIa activity of 0.5 mg of V21 and about 4 times the anti-IIa activity of LMWH. For equivalent anti-Xa activity, V21 has about twice the anti-IIa activity of LMWH,

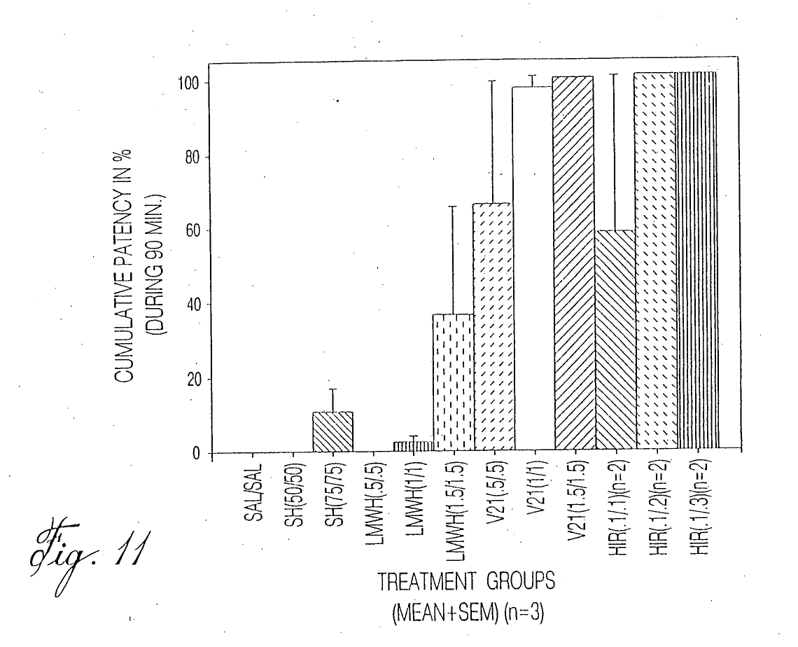

The results obtained during this study are set forth in FIGS. 11, 12 and 13. FIG. 11 compares the efficacy of the four anticoagulants using eumulative time that the aorta remained patent over the 90 minutes of observation as the outcome measure of efficacy. One hundred percent accumulated patency reflects complete patency and 0% cumulative patency reflects immediate and sustained thrombotic occlusion. The stenosed aorta clotted immediately and remained occluded for the full 90 minutes in the control animals, in the rabbits treated with low dose heparin (50/50 unit/Kg) and low dose LMWH (0.5/0.5 mg/Kg). There was a dose response with all four anticoagulants. However, the model was resistant to the antithrombotic effects of heparin and LMWH. Thus, both heparin in a dose of 75/75 units/Kg and LMWH in a dose of 1.0 mg/1.0 mg/Kg were ineffective (percent cumulative patency of 14% and 2% respectively), and LMWH 1.5/1.5 mg/Kg showed only limited effectiveness (38% cumulative patency). In contrast, the model was very responsive to the antithrombotic effects of V21 and hirudin. Thus, V21 at a dose of 0.5/0.5 mg/Kg was more effective than heparin at a dose of 75/75 units/Kg and more effective than LMWH in doses of 1.0/1.0 mg/Kg and 1.5/1.5 mg/Kg. Thus, V21 was at least three fold more potent than LMWH.

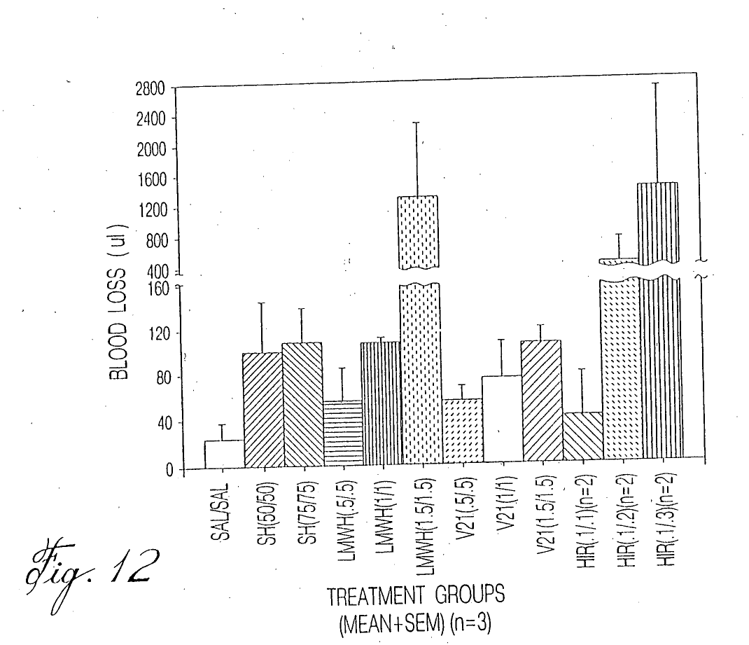

FIG. 12 illustrates the effects of the four anticoagulants on 30 minute blood loss. A dose response was observed with LMWH, V21, and hirudin. At doses that showed greater efficacy, V21 was much safer than LMWH, and at doses that showed equivalent efficacy, V21 was safer than hirudin. V21 was also much more effective than heparin at doses that produced a similar degree of blood loss.

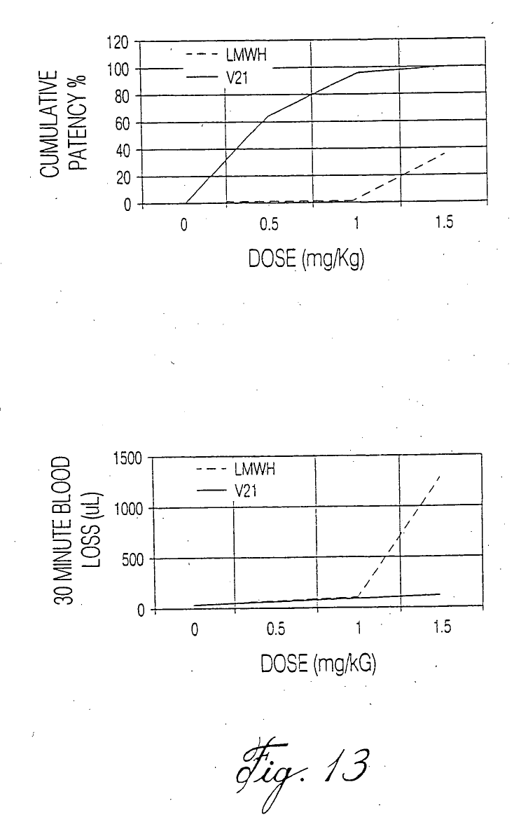

The comparative safety and efficacy of V21 and LMWH is illustrated in FIG. 13. Based on the data (i.e., three animals in each group), V21 appears to about 4 times more potent than LMWH on a weight basis. Therefore, for equivalent anti-Xa activity, V21 is 4 times more potent than LMWH, and for equivalent anti-IIa activity, V21 is about twice as potent. Such data support the importance of fibrin-bound thrombin in promoting thrombogenesis, since V21 is more effective against fibrin-bound thrombin than LMWH or heparin. At doses of 0.5 mg/Kg and 1.0 mg/Kg, V21 appears to be as safe as LMWH (although it is much more effective), but at a dose of 1.5 mg/Kg. LMWH produced much more bleeding than V21. Thus, V21 appears to have a more favorable efficacy to safety profile than LMWH.

### Example 4

### Studies Comparing the MMWH Compositions of the Present Invention (V21) with LMWH

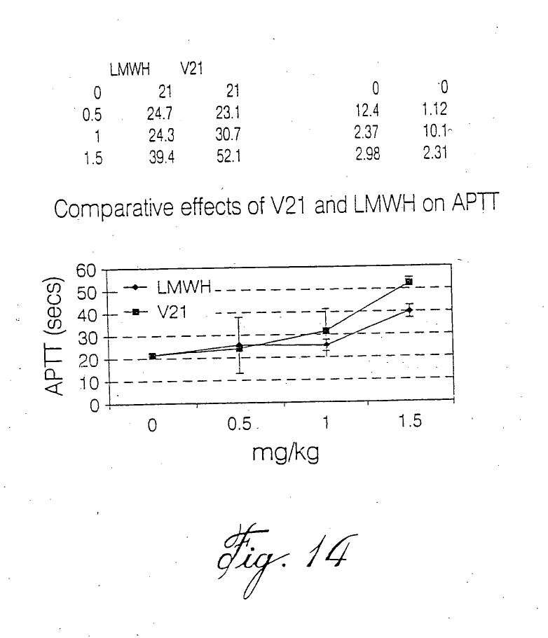

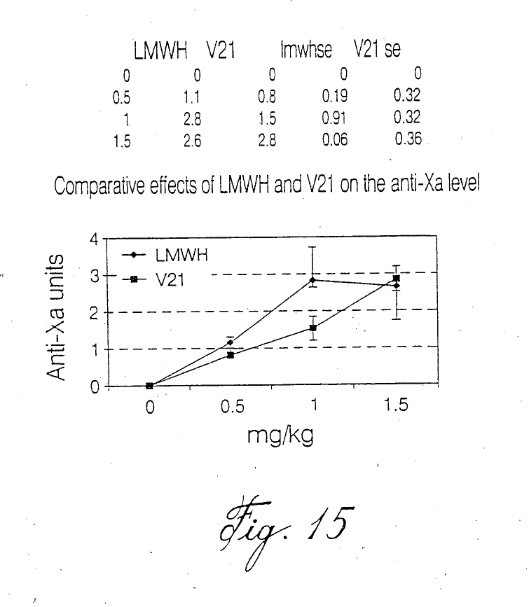

The efficacy of V21 (lot #D32) has been compared with LMWH (Enoxaparin) in both a heparin-sensitive and heparin-resistant thrombosis model in rabbits. The heparin-sensitive model is a venous thrombosis prophylaxis model and the heparin-resistant model is a venous thrombosis treatment model. V21 and LMWH have similar effects ex-vivo on the anti-factor Xa level and on the APTT (FIGS. 14 and 15). Therefore, the two anticoagulants were compared on a gravimetric basis.

### 1.1 Prophylaxis Model

a. Method: Twenty seven male New Zealand White rabbits weighing between three and four kilograms are randomized into 3 treatment groups.
b. Anaesthesia: Anaesthesia is induced by a mixture of intramuscular ketamin (50 mg/kg) and xylazine (2 mg/kg) and maintained by isoflurane (1-3%) and oxygen (1 L/min).
c. Surgical Procedure: The ventral cervical area is shaved and two 22-gauge catheters (Becton-Dickinson, Sandy, UT) are inserted into the left central auricular artery and the marginal auricular vein for blood sampling and for intravenous administration of treatments. The right facial and external jugular vein are exposed through the ventral cervical skin incision. A 2 cm segment of the jugular vein is isolated from surrounding tissues and side branches are ligated using 4-0 silk suture. At this time control arterial blood sample is collected (1.8 ml of blood into 0.2 ml of 3.6% sodium citrate). Blood samples are spun and plasma stored in
−70° C. for blood coagulation studies (A.PTT.TCT. and anti-Xa). Intravenous bolus of 1-125 labelled rabbit fibrinogen (10 μL.-1,000,000.00 CPM) is administered. Thereafter rabbits are randomized to one of the following iv treatments:
   1. Saline (n=9) iv bolus of 1 ml of saline
   2. Low molecular weight heparin (n=9)(Lovenox, enoxaparin sodium, lot #923, Rhone-Poulenc Rorer, Montreal, Quebec, Canada) in a dose of 0.50 mg/kg (n3), 1.00 mg/kg (n3), 1.5 mg/kg (n=3).
   3. V-21 (n=9) (D-32, lot #521982132) in a dose of 0.50 mg/kg (n=3), 1.00 mg/kg (n=3), and 1.5, mg/kg (n=3).

Four minutes after the treatment administration the right jugular vein is damaged in the length of 2 cm by 15 passages of the inflated balloon catheter (#4, Fogarty thrombectomy catheter). The balloon catheter is introduced into the right jugular vein via the right facial vein. Right after balloon vein injury, the catheter is withdrawn and a second arterial blood sample is taken. In addition, 1 ml of blood is also collected to measure radioactivity. Blood stasis is then induced within the 2 cm right jugular vein segment by placing two tourniquets around the vein. After 15 minute occlusion the jugular vein segment is excised and opened onto a pre-weighed square and weighed. Thereafter, the third arterial blood sample is collected for blood coagulation assay analysis.
d. End-Points:

Clot weight (%) s calculated as a percentage of blood by weight trapped in the venous segment. Clot radioactivity (%) is calculated as a percentage of 1 ml of whole blood radioactivity. Plasma samples were analyzed for APTT, TCT and anti-Xa.

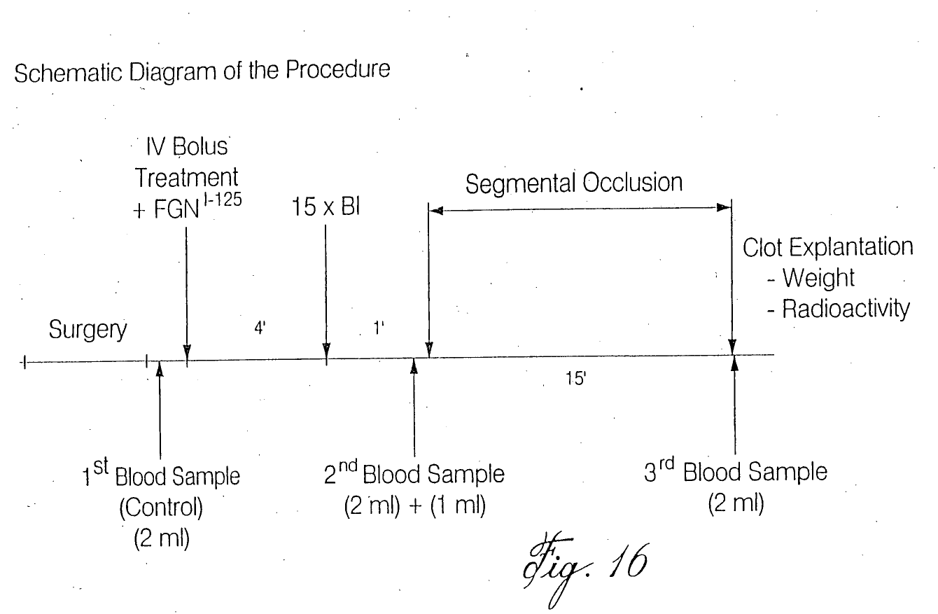

Schematic diagram of the procedure is shown in appended FIG. 16.
e. Results:

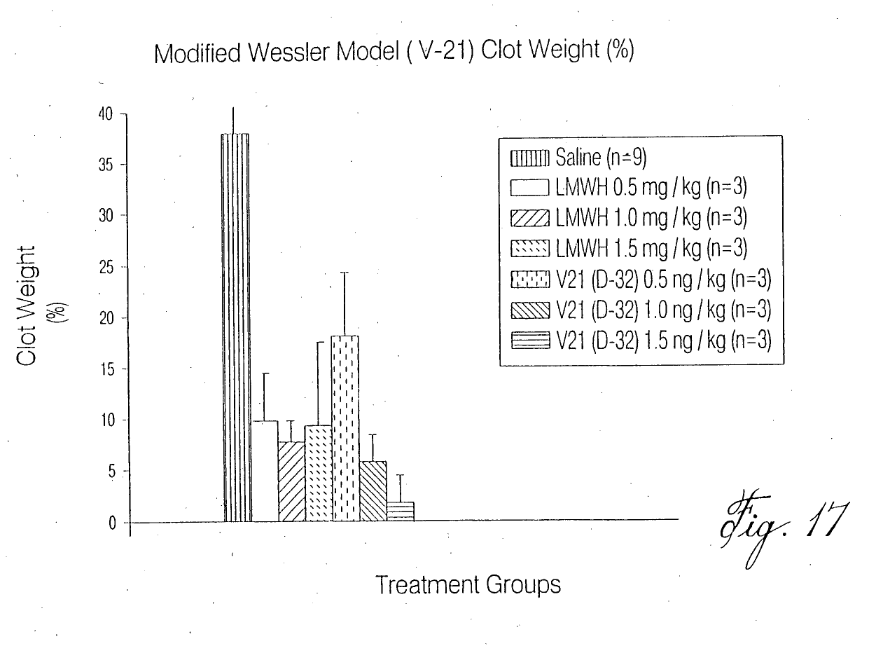

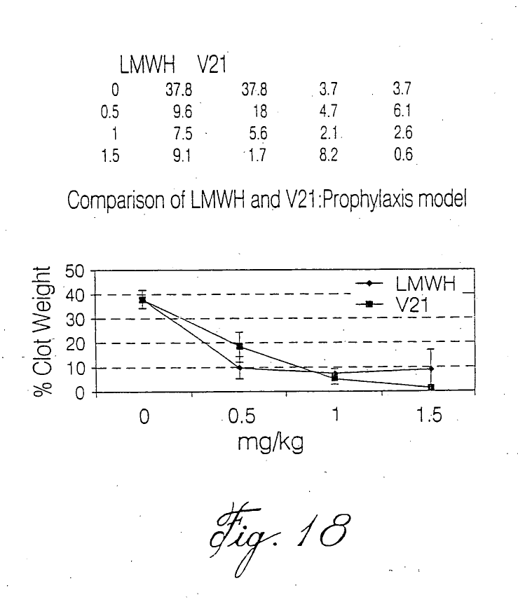

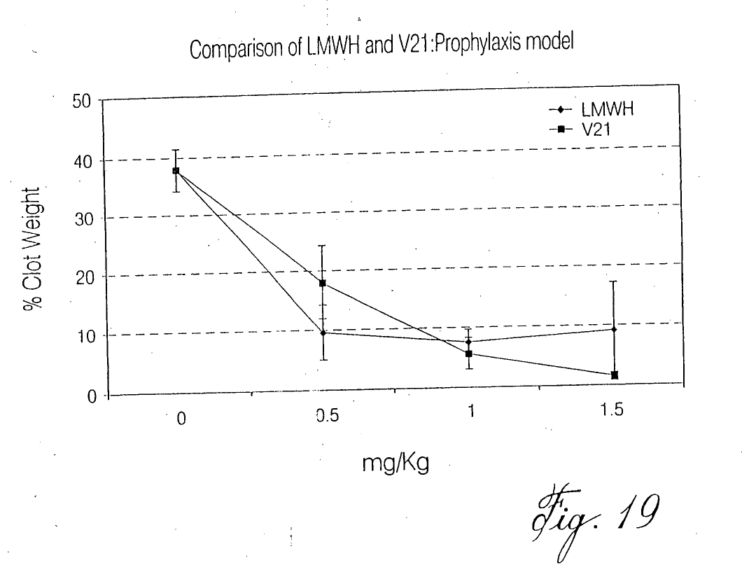

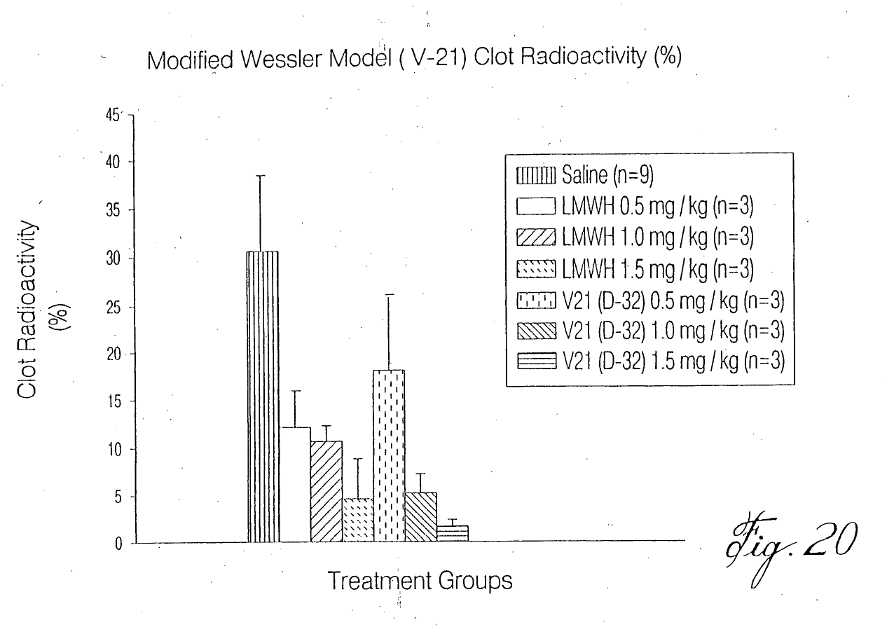

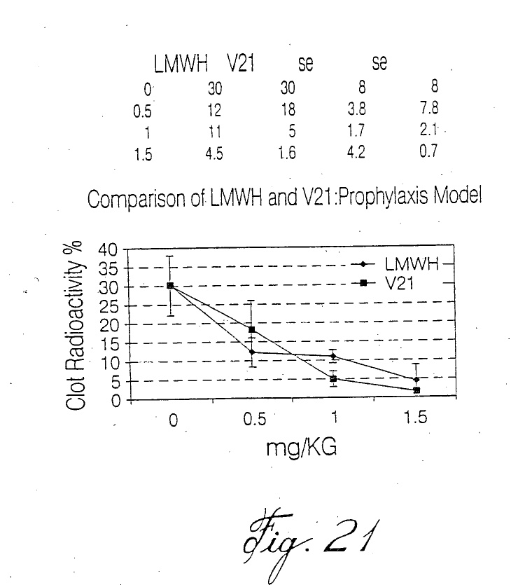

As shown in FIGS. 16, 17 and 18 (for clot weight) and FIGS. 19, 20 and 21 (clot radioactivity), both agents are effective in this heparin-sensitive model, but V21 produces a sleeper dose response and in more effective than LMWH at the two higher doses.

### 1.2 Treatment of Deep Vein Thrombosis in Rabbit Model

e. 24 Hour Follow-up

The purpose of this study was to compare the efficacy of V-21 with LMWH both administered subcutaneously in the rabbit model of deep vein thrombosis.

Twenty four specific pathogen free, New Zealand White, male rabbits (3-4 kg of b, wt.) were anesthetized by intramuscular injection of ketamin (50 mg/kg) and xylazine (2 mg/kg). The ventral cervical area was shaved and prepped with alcohol and iodine solution. Venous and arterial catheters were inserted into the left central auricular artery and the marginal auricular vein using an 22 gauge intravenous catheter (Angiocath, Becton Dickinson Vascular Access, Sandy, Utah., USA) for blood sample collection, and for intravenous administration of fluids and anticoagulants. Rabbits were transferred into an operating room and maintained on inhalation anesthesia which consisted of a mixture of isoflurane (1-4%), oxygen (1 L/min) and nitrous oxide (0.5 L/min) delivered by a face mask.
b. Clot Formation: The right external jugular and the facial vein were exposed through the ventral cervical skin incision. Segmental occlusion of the facial vein was achieved by two No 4-0 silk sutured placed 0.5 centimetres apart. All side branches of the jugular vein were ligated in the length of 4 centimetres. Fogerty thrombectomy catheter (#4 Fr) was introduced into the jugular vein via the facial vein and inflated. Four centimetres of the jugular vein was damaged by 15 passages of inflated balloon catheter and than the catheter was withdrawn. A 1.5 centimetres occluded jugular vein segment was created using two 4-0 silk sutures placed around the damaged vein and then emptied using finger compression. One millilitre of arterial blood was drawn from the central auricular artery into the 1 ml syringe and mixed in a sterile tube with approximatley 1 μCi of iodine-125 labelled rabbit fibrinogen. 0.6 millilitres of the radiolabelled blood was then drawn from the tube into a 1 ml syringe and the first 0.4 ml of labelled blood was equally divided into two tubes and left to clot. The remaining 0.2 ml was then injected into the occluded jugular vein segment via the home made cannula (23 gauge needle connected to PB #60). Clots generated in the test tubes served as baseline values for clot weight and radioactivity. Pilot studies have shown that there was around 5% difference in clot weight or radioactivity between the clots generated in tubes and in the jugular vein. The thrombus generated in the jugular vein was left to mature for 30 minutes and the facial vein was ligated. Twenty five minutes into the thrombus maturation rabbits were randomized to receive:
   1) saline treatment (n=4) 1 ml of sterile saline BID sc;
   2) low molecular weight heparin (Enoxaparin sodium, Lovenox lot #923, Rhone-Poulenc Rorer, Montreal, Quebec) at a dose of 1 mg/kg BID, sc (n=4), and 3 mg/kg BID sc (n=4); and
   3) V-21 (D-32, lot #521982132) at a dose of 1 mg/kg BID sc (n=4) and at 3 mg/kg BID sc (n=4).

Thirty percent of the first dose was administered intravenously and 70% subcutaneously; the second dose was given only subcutaneously. Just prior to tourniquet removal at 30 minutes, the thrombus was fixed to the vein wall by two silk sutures to prevent its migration in the post-operative period. There was no residual stenosis of the jugular vein left after tourniquet removal. The cervical incision was closed in a routine manner. Rabbits were left to recover breathing 100% oxygen and then transferred to the recovery room. All rabbits were euthanized at the 24 hour time interval.
c. Blood collection: Arterial blood was collected prior to surgery (control) and at 5 minutes, 1, 3, 6, 9, 12, and 24 hours after clot maturation. At each time interval 2 millilitres of citrated blood was collected (9:1 ratio, 3.8% sodium citrate) for APTT, TCT and Xa assays. Blood loss was replaced by iv administration of saline.
d. End-points: Using thrombus weight in milligrams (AG Balances #104m Mettler Toledo, Fisher Scientific Limited, Whitby, Ontario) and thrombus radioactivity (CPM) at the time of clot induction (clot created in tubes) and at 24 hours the following end-points were calculated:

Percentage change in clot weight (PCCW) was calculated using clot weight at 24 hours minus clot weight generated in a test tube at the time of surgery divided by clot weight generated in a test tube times 100.

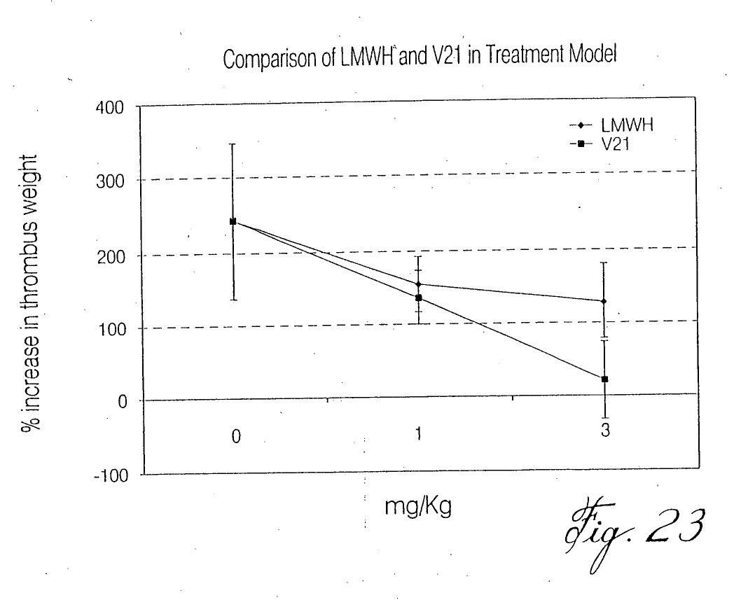

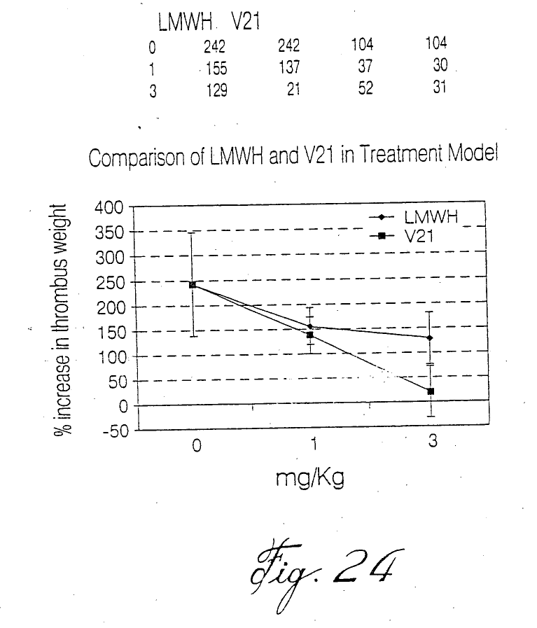

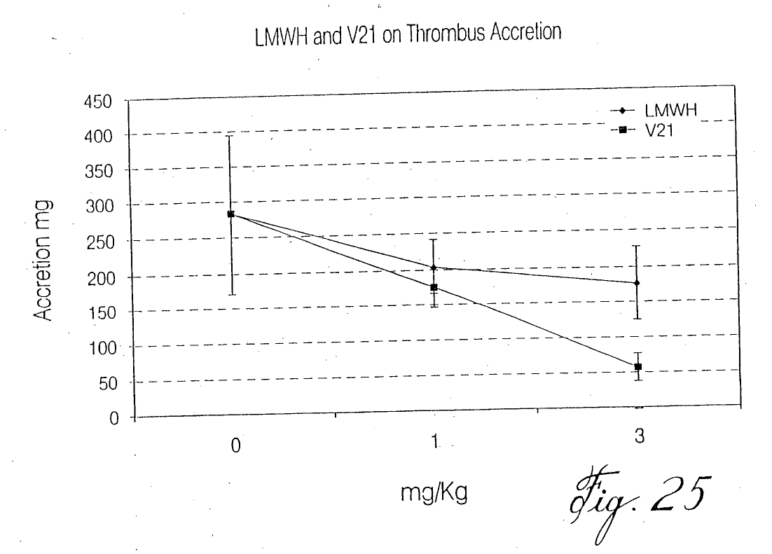

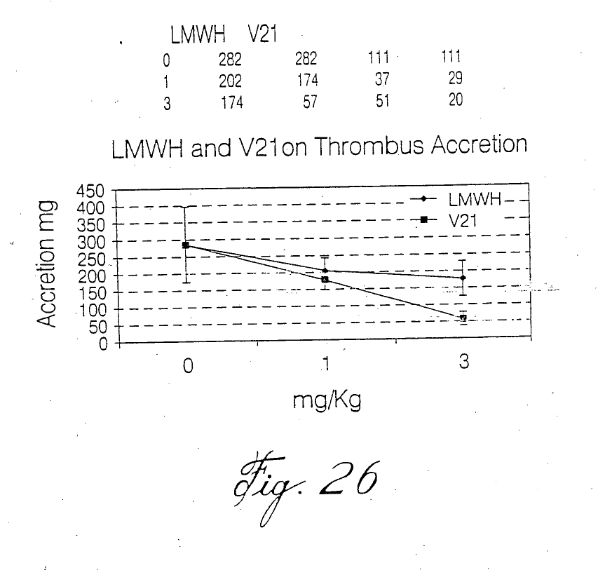

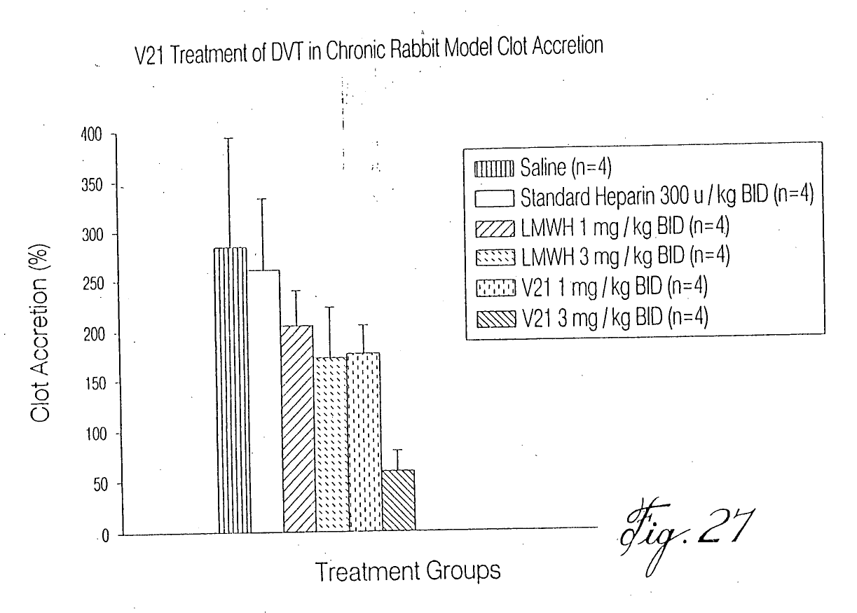

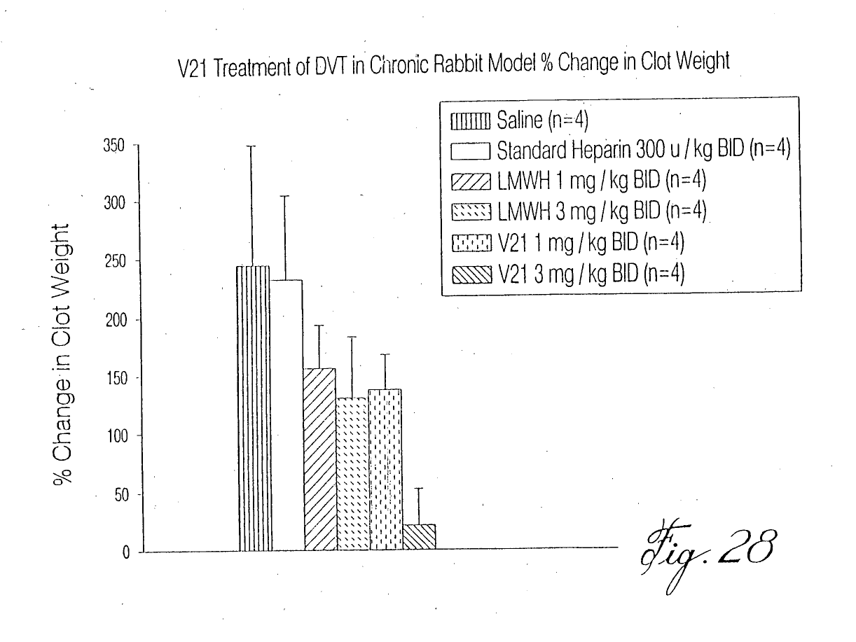

Clot accretion (CA) at % was calculated as follows: AC=PCCW−CL
e. Results: As illustrated in FIGS. 23-28, V21 is more effective than LMWH in this heparin-resistant model.

### Example 5

### Preparation of the MMWH Compositions of the Present Invention by a Limited Periodate Oxidation/Hydrolysis of Heparin

### 1.1 Study of Limited Periodate Oxidation/Hydrolysis of Heparin

Heparin was dissolved in deuterated water to make 10% of stock solution. Sodium periodate was dissolved in deuterated water to make 100 mM stock solution and kept at 4° C. The periodate oxidation reaction was carried out at 2.5% of heparin concentration with increasing sodium periodate concentration, 1 mM, 2.5 mM, 5 mM, 8 mM, 10 mM, and 20 mM, at room temperature for about 18 hours. The reaction was stopped by adding 50 mM of ethylene glycol and incubation for 30 minutes. Then, the reaction mixture was brought to 0.25 N NaOH and incubated at room temperature for 3 hours. After the reaction, the pH was adjusted to pH 7 by 6 N HCl. An aliquot of each reaction mixture was run on an HPLC-GPC (G2000 column, 0.5 ml/min, injection volume 20 μl) for molecular weight analysis. The molecular weight profiles of the reaction at sodium periodate concentrations of 5 mM, 8 mM, 10 mM, and 20 mM decrease in comparison to heparin with increasing sodium periodate concentration. The result indicated that the desired cleavage can be achieved using sodium periodate concentrations of between about 5 mM and about 20 mM, and at room temperature for about 18 hours. The study (not shown) indicated that the best alkaline hydrolysis an be achieved using 0.25 N NaOH, at room temperature for 3 hours. Thus, the reaction conditions used in this experiment are called “limited periodate/hydrolysis” conditions.

### 1.2 Preparation of MMWH compositions of the Present Invention by Limited Periodate Oxidation/Hydrolysis

100 mg of heparin was treated using the limited periodate/hydrolysis conditions, 7 mM sodium periodate, and purified by P30 gel-filtration chromatography. 30 mg of final product, i. e., V21-D32, was obtained having a molecular weight ranging from about 6,000 Daltons to about 12,000 Daltons, and having a peak molecular weight of about 9,000 Daltons.

### Example 6

Studies were undertaken to select MMWH compositions with an optimal molecular weight range and to identify a manufacturing process which could be readily scaled up to obtain a heparin fraction within this range.

A molecular weight range between 6,000 and 10,000 was selected as an optimal molecular weight range. Compositions with a minimum molecular weight of 6,000, which corresponds to 20 saccharide units, should provide heparin chains that heave pentasaccharide-containing chains long enough to bridge antithrombin to thrombin. With a maximum molecular weight of 10,000, which corresponds to 33 saccharide units, the chains will (a) be too short to bridge thrombin to fibrin, a phenomenon that requires chains of 40 saccharide units or more, and (b) too short to exhibit non-specific binding to plasma proteins, a phenomenon that occurs with chains of 30 saccharide units or more.

### Heparin Fractions:

Unfractionated heparin was depolymerized with heparinase, nitrous acid, or periodate to yield fractions of approximately 6,000, 8,000, and 10,000 Da. While initial fractions produced by these three methods were polydispersed, more size-restricted fractions of these molecular weights were prepared using either heparinase or nitrous acid depolymerization. The characteristics of these fractions are illustrated in Table 1 along with their specific anti-Xa and anti-IIa activities.

### Affinities for Antithrombin

The affinities of each of the heparin fractions for antithrombin was determined as previously described (Weitz et al, Ciruculation 1999:99:682-689). Briefly, a 1×1 cm, quartz cuvette containing 100 nM antithrombin in 2 ml of 20 mM Tris-HCl, pH 7.4 150 mM NaCl (TBS) was excited at 200 nm (6-nm slit width) and intrinsic fluorescence was continuously monitored in time drive at 340 nm (6-nm slit width) with a Perkin-Elmer LS50B luminescence spectrometer. The contents of the cuvette were stirred with a micro-stir bar and maintained at 25° C. with a recirculating water bath. Intrinsic fluorescence intensity was measured before (Iₒ) and after (I) addition of 5 to 10 ml of 10-mg/ml solutions of the various heparin fractions. Titrations were continued until there was no change in I. After the experiment, I values were read from the time drive profile and IIIₒ values were calculated and plotted versus heparin concentration. The data were then analyzed as described previously (Weits et al, supra). From this analysis, stoichiometry can be obtained which is interpreted as indicating the proportion of pentasaccharide-containing chains within each heparin fraction.

The affinities are summarized in Table 2. Also illustrated is the estimated percentage of pentasaccharide-containing chains within each fraction. Fractions prepared by either heparinase or nitrous acid depolymerization exhibit similar affinities for antithrombin. Although the 10,100 Da fraction prepared by periodate depolymerization exhibits affinity for antithrombin similar to that of heparinase or nitrous acid-derived fractions, the lower molecular weight periodate-derived fractions have lower affinities consistent with their reduced anti-IIa and anti-Xa activities (Table 1). As might be expected, regardless of the method used for depolymerization, the percentage of pentasaccharide-containing chains increases as the mean molecular weight increases.

As controls for these analyses, unfractionated heparin, high and low affinity fractions of heparin prepared by affinity chromatography using an antithrombin column, enoxaparin, and synthetic pentasaccharide also were studied. As illustrated in Table 3, the high affinity fraction of heparin and synthetic pentasaccharide exhibit the highest affinity for antithrombin. Only these two preparations have 100% pentasaccharide-containing chains.

### Affinities for Thrombin:

The affinities of the polydispersed heparinase, nitrous acid and periodate-derived heparin fractions for thrombin were measured as described above except thrombin was used in place of antithrombin (Fredenburgh, J C et al. J. Biol. Chem. 1997:272:25493-25499). As illustrated in Table 4, when affinities are expressed in μg/ml, all fractions exhibited similar affinities for thrombin.

### Heparin-catalyzed Rates of Thrombin Inhibition by Antithrombin in the Absence or Presence of Fibrin Monomer:

The second order rate constants for thrombin inhibition by antithrombin were measures in the absence or presence of the various heparin fractions in concentrations ranging from 0 to 600 μg/ml. Heparin-catalyzed rates of thrombin inhibition by antithrombin were measured both in the absence or presence of 4 μM fibrin monomer. The fibrin monomer was prepared as previously described, and the data were analyzed as described elsewhere (Becker D L et al. J. Biol. Chem. 1999:274:6226-6233)

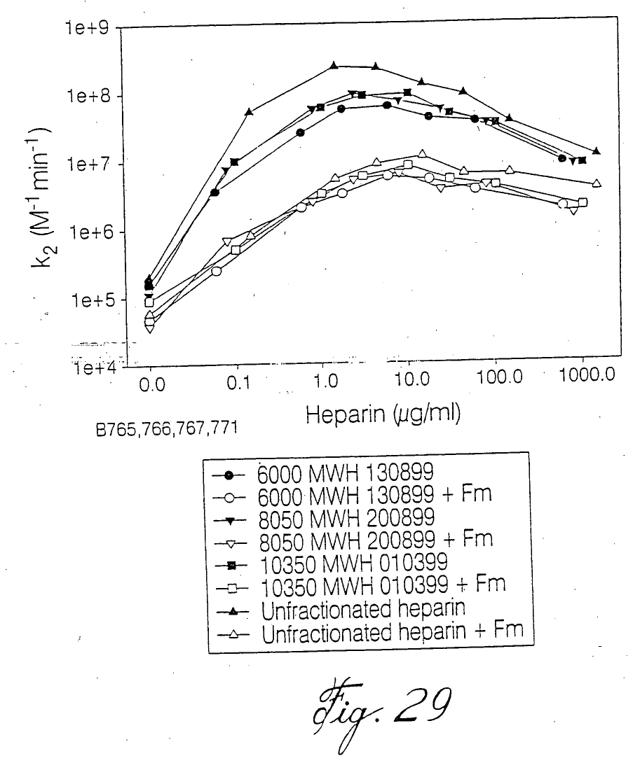

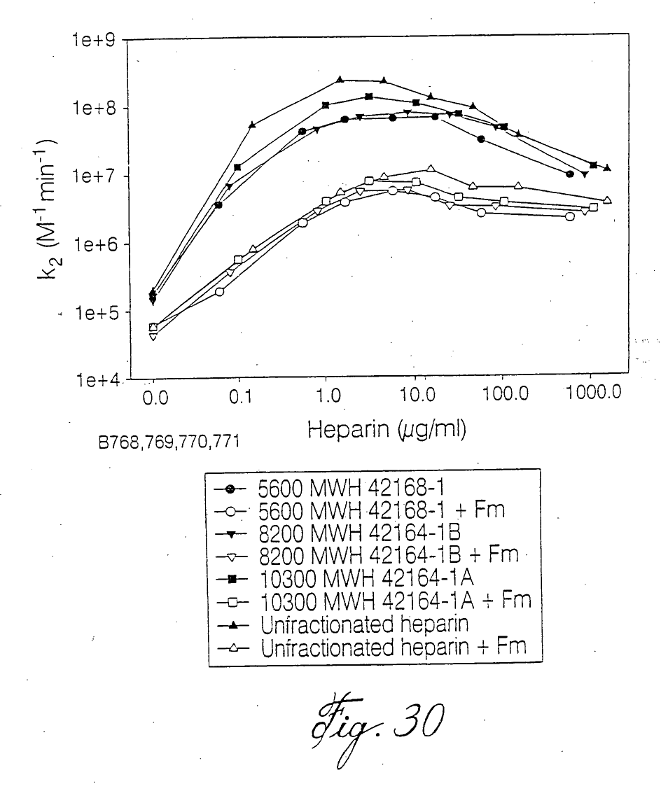

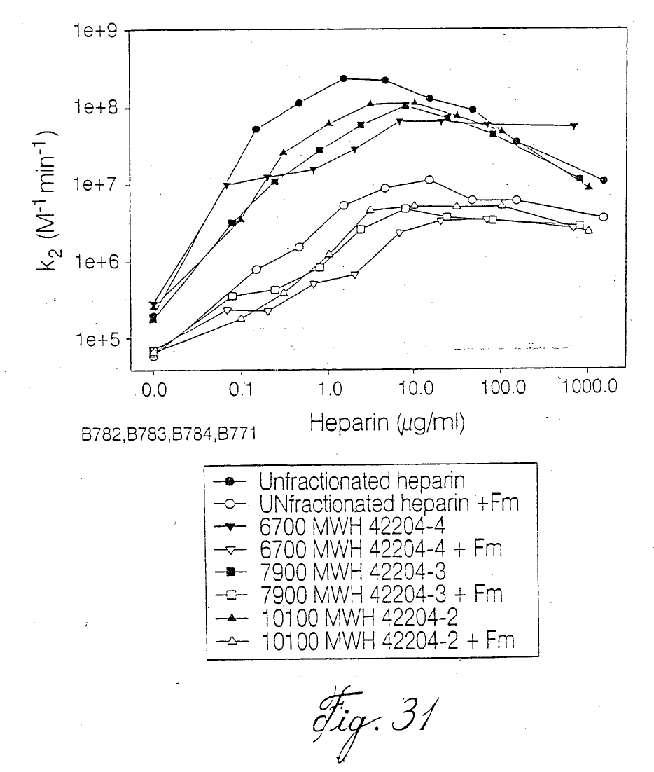

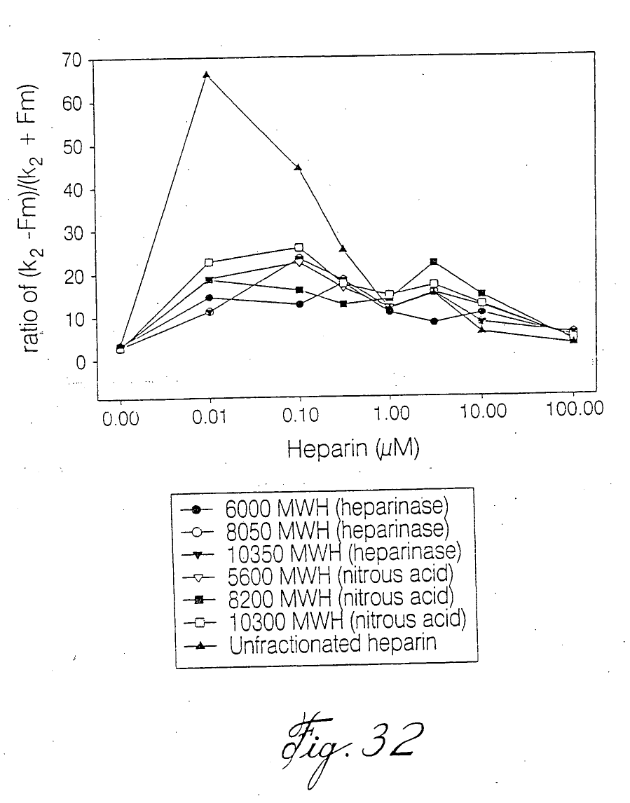

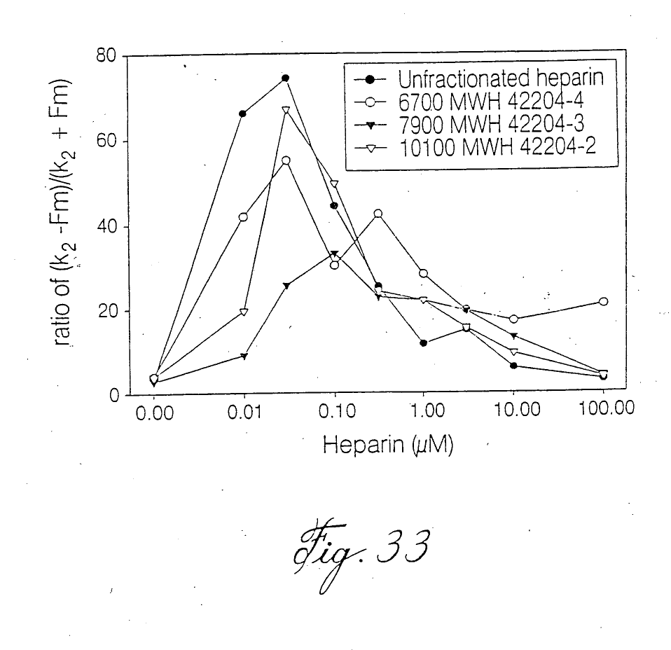

The inhibitory effect of fibrin-monomer on the rates of inhibition of thrombin by antithrombin is shown with the heparinase (FIG. 29), nitrous acid (FIG. 30), and periodate-derived heparin fractions (FIG. 31). The background inhibition with fibrin monomer is 6-fold as determined by measuring the inhibitory effect of fibrin monomer on the heparin-catalysed rate of factor Xa inactivation by antithrombin, (FIGS. 32 and 33). There is less reduction in the rate of thrombin inactivation by antithrombin with the heparinase or nitrous acid-derived heparin fractions than with unfractionated heparin. In contrast, greater inhibition with fibrin monomer is seen with the periodate-derived heparin fractions (FIG. 31). With the size-restricted heparinase-derived fractions, fibrin-monomer produces no more than background inhibition.

### Heparin-catalyzed Inhibition of Factor Xa by Antithrombin:

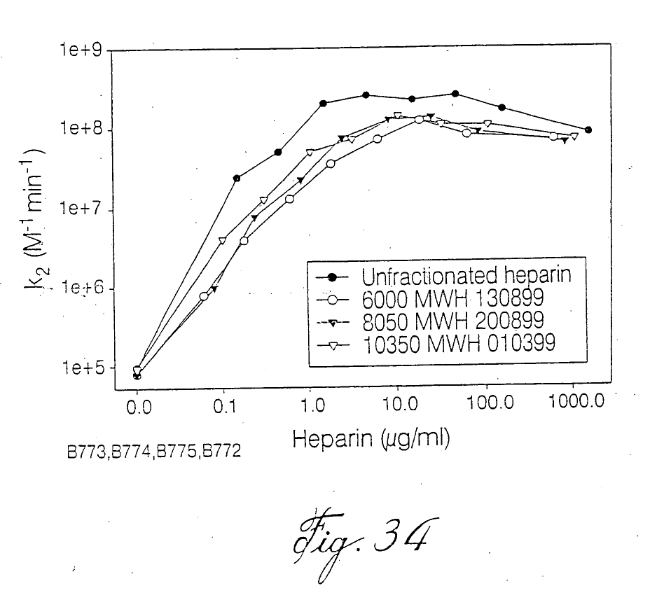

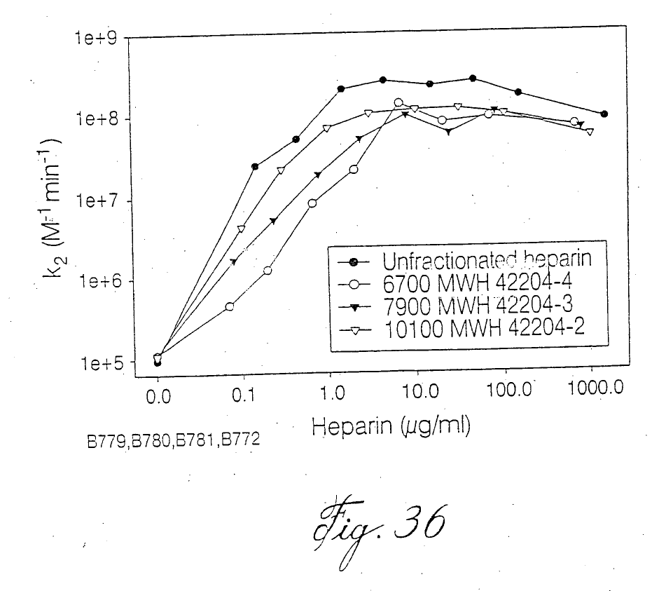

The second order rate constants for factor Xa inhibition by antithrombin were measured in the absence or presence of the various heparin fractions in concentrations ranging from 0 to 1,500 μg/ml as described elsewhere (Becker et al, supra). The results for the heparinase, nitrous acid, and periodate-derived fractions are illustrated in FIGS. 34 and 36, respectively. When added in gravimetrically equivalent amounts, all of the heparin fractions produce less catalysis of factor a inhibition by antithrombin than unfractionated heparin.

### Augmentation of Thrombin Binding to Fibrin:

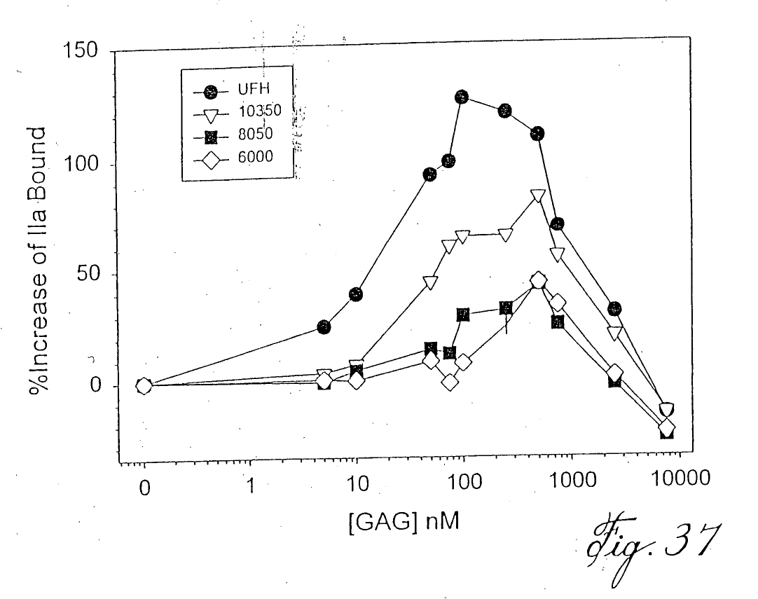

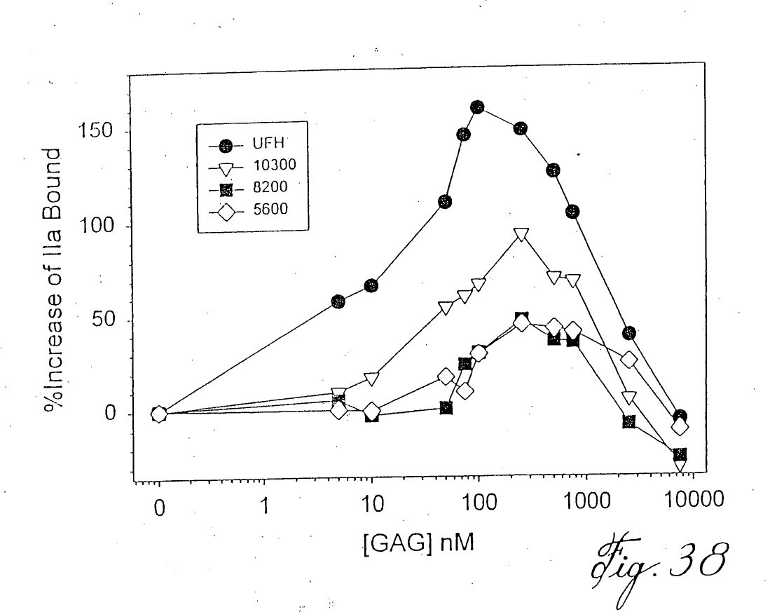

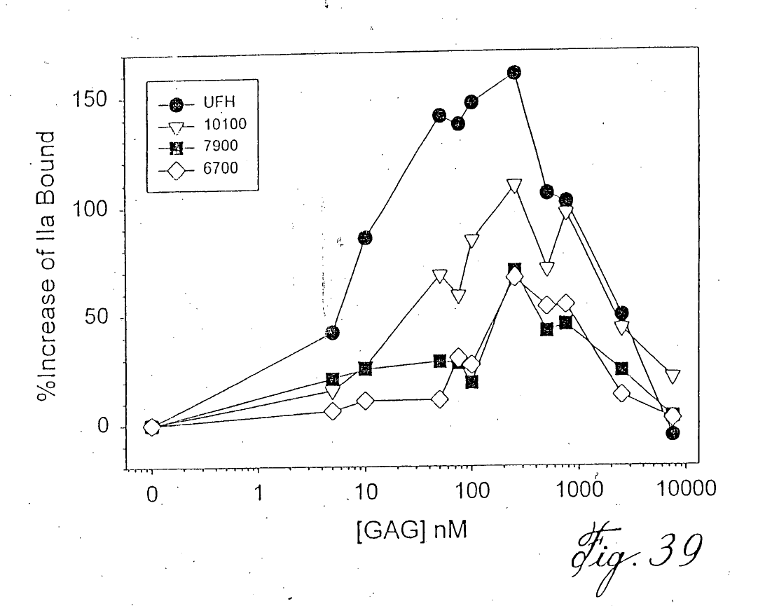

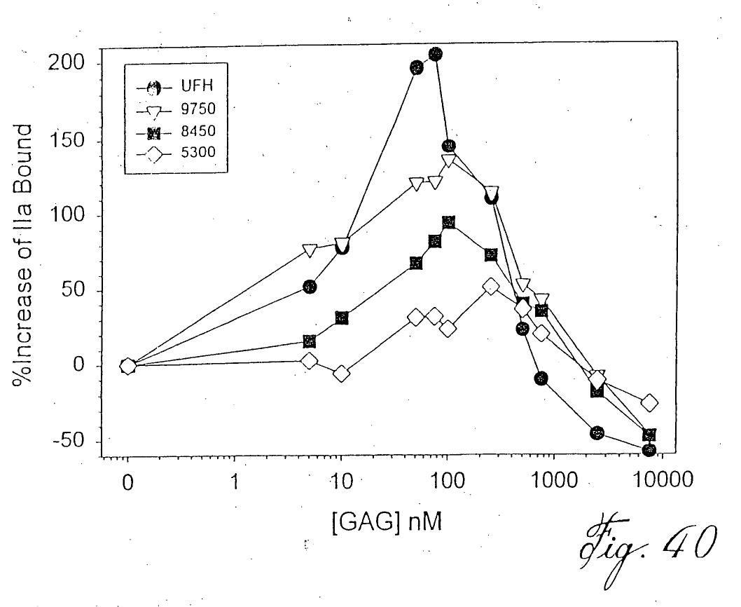

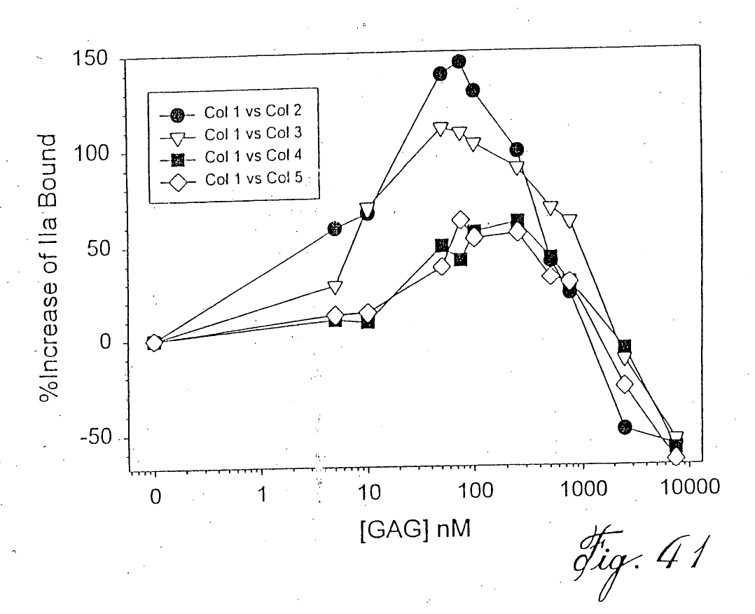

I¹²⁵-labeled thrombin binding to fibrin was measured in the absence or presence of the various heparin fractions in concentrations ranging from 0 to 7,500 nM as previously described (Becker et al, supra). Unfractionated heparin was used as a control in these experiments. The results with heparinase, nitrous acid, and periodate-derived heparin fractions are illustrated in FIGS. 37 to 39, respectively. Regardless of the method of depolymerization, the 10,000 Da fractions augment thrombin binding to fibrin to a greater extent than the lower molecular weight fractions. This is best illustrated with the more size-restricted heparinase or nitrous acid-derived fractions (FIGS. 40 and 41, respectively).

### Antithrombotic Activity of Heparin Fractions:

An extracorporeal circuit was used to compare the antithrombotic activity of the heparin fractions. As previously described (Weitz et al, supra), different concentrations of each of the heparin fractions was added to recalcified human whole blood spiked with ¹²⁵I-labeled human fibrinogen and maintained at 37° C. in a water bath. A peristaltic pump was then used to circulate the blood through a 40μ blood filter. Clotting of blood within the filter was detected by (a) measuring pressure proximal to the filter with an in-line pressure gauge, and (b) removing serial blood samples from the reservoir and counting residual radioactivity as an index of fibrinogen consumption. Starting activated clotting times also were measured.

As illustrated in Table 5, regardless of the method of depolymerization, fractions of 10,000 Da were effective at a concentration of 10 μg/ml. Thus, filter patency was maintained during the 90 min observation period and fibrinogen consumption was less than 10%. At a concentration of 10 μg/ml, the heparinase-derived 8,000 Da fraction was effective. The 6,000 Da heparinase fraction was effective at 14 μg/ml. Although patency was maintained with 14 or 16 82 g/ml of the 5,600 da nitrous acid-derived fractions, fibrinogen consumption was 33 and 20%, respectively. As a control, enoxaparin was also evaluated. This drug was ineffective at 10 or 20 μg/ml with filter failure occurring at 30 and 55 min. respectively.

The antithrombotic activities of the more size-restricted heparinase and nitrous acid-derived heparin fractions are illustrated in Table 6. All fractions were tested at a concentration of 10 μg/ml enoxaparin serving as a control. Except for the 5,300 Da heparinase-derived fraction, all of the heparin fractions were effective in maintaining patency for >90 min and reducing fibrinogen consumption to <10%. In contrast, enoxaparin was ineffective with filter failure occurring at 30 min and fibrinogen consumption of 73%. The antithrombotic activities of the more size-restricted heparinase, nitrous acid-derived heparin and periodate fractions are illustrated in Table 7. All fractions were tested at a concentration of 10 μg/ml with 10 μg/ml enoxaparin serving as a control. The heparinase and nitrous acid derivative fractions were effective in aminating patency. The periodate-derived fraction and enoxaparin were less effective.

It is to be understood that the above description is intended to be illustrative and not restrictive. Many embodiments will be apparent to those of skill in the art upon reading the above description. The scope of the invention should, therefore, be determined not with reference to the above description, but should instead be determined with reference to the appended claims, along with the full scope of equivalents to which such claims are entitled. The disclosures of all articles and references, including patent applications and publications, are incorporated herein by reference in their entirety for all purpose.

TABLE 1
CHARACTERISTICS OF THE HEPARIN
FRACTIONS PROVIDED BY LEO

| Depolymerization | Molecular | Poly- | Anti- | Anti- |
|---|---|---|---|---|
| Method | Weight | dispersity | IIa | Xa |
| Heparinase | 6,000 | 1.5 | 72 | 106 |
| | 8,500 | 1.5 | 100 | 134 |
| | 10,350 | 1.5 | 152 | 111 |
| HN 0₂ | 5,600 | 1.5 | 59 | 118 |
| | 8,200 | 1.4 | 100 | 152 |
| | 10,300 | 1.4 | 119 | 180 |
| IO₄⁻ | 6,700 | 1.5 | 11 | 30 |
| | 7,900 | 1.5 | 19 | 43 |
| | 10,100 | 1.5 | 43 | 88 |
| | 10,300 | 1.5 | 42 | 84 |
| Heparinase | 5,300 | 1.2 | 22 | 81 |
| | 8,450 | 1.2 | 67 | 116 |
| | 9,750 | 1.3 | 87 | 155 |
| HN0₋ | 5,900 | 1.2 | 32 | 95 |
| | 7,700 | 1.3 | 84 | 123 |
| | 9,300 | 1.2 | 106 | 162 |

TABLE 2
AFFINITIES OF HEPARIN FRACTIONS FOR ANTITHROMBIN
AND PERCENTAGE OF PENTASACCHARIDE-CONTAINING
CHAINS IN EACH FRACTION

| | | |
|---|---|---|
| | K_{d} | Pentasaccharide- |
| Glycosaminoglycan | nM | containing % |

| | | | |
|---|---|---|---|
| Heparinase | 6,000 | 91.2 ± 15.9 | 14.8 |
| | 8,050 | 61.7 ± 4.2 | 20.8 |
| | 10,350 | 48.0 ± 7.8 | 27.0 |
| HN0₂ | 5,600 | 55.6 ± 0.2 | 16.0 |
| | 8,200 | 42.5 ± 9.1 | 25.2 |
| | 10,300 | 37.5 ± 2.9 | 31.5 |
| IO₄⁻ | 6,700 | 170.1 ± 27.4 | 6.8 |
| | 8,200 | 140.3 ± 4.5 | 10.5 |
| | 10,300 | 57.0 ± 28.3 | 25.6 |
| Heparinase | 5,300 | 421.6 ± 72.3 | 15.9 |
| | 8,450 | 167.0 ± 17.0 | 29.4 |
| | 9,750 | 138.4 ± 2.2 | 32.2 |
| HN0₂ | 5,900 | 32.8 ± 0.3 | 21.1 |
| | 7,700 | 23.1 ± 4.1 | 26.9 |
| | 9,300 | 17.0 ± 0.3 | 36.6 |

TABLE 3
AFFINITIES OF UNFRACTIONATED HEPARIN, HEPARIN
WITH HIGH OR LOW AFFINITY FOR ANTITHROMBIN,
ENOXAPARIN AND SYNTHETIC PENTASACCHARIDE FOR
ANTITHROMBIN AND PERCENTAGE OF PENTASACCHARIDE-
CONTAINING CHAINS IN EACH PREPARATION

| | K_{d} | Pentasaccharide- |
|---|---|---|
| Glycosaminoglycan | nM | containing % |
| Unfractionated heparin | 31.7 | 43.1 |
| High affinity heparin | 10.7 | 114 |
| Low affinity heparin | 6670.0 | 1.0 |
| Enoxaparin | 46.8 | 14.4 |
| Pentasaccharide | 31.0 | 102 |

TABLE 4
AFFINITIES OF HEPARINASE AND NITROUS ACID-DERIVED
HEPARIN FRACTIONS FOR THROMBIN

| | | |
|---|---|---|
| | K_{d} | K_{d} |
| Glycosaminoglycans | nM | μg/ml |

| | | | |
|---|---|---|---|
| Heparinase | 6,000 | 1517 ± 196 | 9.1 ± 1.2 |
| | 8,050 | 872 ± 9 | 7.0 ± 0.1 |
| | 10,350 | 699 ± 97 | 1.2 ± 1.0 |
| HN0₂ | 5,600 | 1288 ± 92 | 7.2 ± 0.5 |
| | 8,200 | 695 ± 37 | 5.7 ± 0.3 |
| | 10,300 | 632 ± 51 | 6.5 ± 0.5 |
| IO₄ | 6,700 | 731 ± 159 | 4.9 ± 1.1 |
| | 7,900 | 587 ± 8 | 4.6 ± 0.1 |
| | 10,100 | 285 ± 5 | 2.9 ± 0.5 |

TABLE 5
ANTITHROMBOTIC ACTIVITY OF HEPARINASE, NITROUS
ACID, AND PERIODATE-DERIVED HEPARIN FRACTIONS
AND ENOXAPARIN IN AN EXTRACORPOREAL CIRCUIT

| | | | | Time to | | |
|---|---|---|---|---|---|---|
| | | | Concen- | Filter | Fibrinogen | Starting |
| | | | tration | Failure | Consumption | ACT |
| Glycosaminoglycan | | | μg/ml | min | % | sec |
| Heparinase | 6 | kDa | 8 | 75 | 82 | 271 |
| | | | 10 | >90 | 68 | 248 |
| | | | 12 | >90 | 31 | 226 |
| | | | 14 | >90 | 7 | 335 |
| | 8 | kDa | 5 | 40 | 70 | 241 |
| | | | 6 | 45 | 70 | 230 |
| | | | 8 | >90 | 54 | 256 |
| | | | 10 | >90 | 6 | 282 |
| | 10 | kDa | 5 | 45 | 72 | 231 |
| | | | 6 | >90 | 36 | 299 |
| | | | 8 | >90 | 29 | 300 |
| | | | 10 | >90 | 4 | 327 |
| HN0₂ | 5.6 | kDa | 10 | >90 | 29 | 238 |
| | | | 12 | >90 | 57 | 235 |
| | | | 14 | >90 | 33 | 238 |
| | | | 16 | >90 | 20 | 264 |
| | 8.2 | kDa | 10 | 60 | 81 | 239 |
| | | | 11 | >90 | 28 | 313 |
| | | | 12 | >90 | 10 | 301 |
| | | | 16 | >90 | 8 | 428 |
| | 10.3 | kDa | 8 | >90 | 14 | 303 |
| | | | 10 | >90 | 28 | 287 |
| | | | 11 | >90 | 7 | 341 |
| | | | 12 | >90 | 7 | 359 |
| IO₄⁻ | 6.7 | kDa | 10 | 45 | 68 | — |
| | 7.9 | kDa | 10 | 90 | 73 | 299 |
| | 10.1 | kDa | 10 | >90 | 6 | 318 |
| Enoxaparin | | | 10 | 30 | 73 | 202 |
| | | | 20 | 55 | 64 | 231 |

TABLE 6
COMPARISON OF ACTIVITY OF 10 μg/ml HEPARINASE,
NITROUS ACID-DERIVED HEPARIN FRACTIONS WITH
ENOXAPARIN IN EXTRACORPOREAL CIRCUIT

| | | Time to Filter | Fibrinogen | Starting |
|---|---|---|---|---|
| | | Failure | Consumption | ACT |
| Glycosaminoglycan | | min | % | sec |
| Heparinase | 5,300 | 30 | 80 | 205 |
| | 8,450 | >90 | 9 | 283 |
| | 9,750 | >90 | 8 | 312 |
| HN0₂ | 5,900 | >90 | 11 | 278 |
| | 7,700 | >90 | 5 | 314 |
| | 9,300 | >90 | 8 | 557 |
| Enoxaparin | | 30 | 73 | 202 |

TABLE 7
COMPARISON OF ACTIVITY OF 10 μg/ml HEPARINASE, NITROUS
ACID-DERIVED AND PERIODATE-DERIVED HEPARIN FRACTIONS
WITH ENOXAPARIN IN EXTRACORPOREAL CIRCUIT

| | | Time to Filter | Fibrinogen | Starting |
|---|---|---|---|---|
| | | Failure | Consumption | ACT |
| Glycosaminoglycan | | min | % | sec |
| Heparinase | 8,450 | >90 | 7.1 | 289 |
| HN0₂ | 7,700 | >90 | 5.6 | 296 |
| IO4 | 7,900 | 30 | 77 | 242 |
| Enoxaparin | | 30 | 80 | 232 |

1-34. (canceled)

35. A medium molecular weight heparin (MMWH) composition comprising a mixture of sulfated oligosaccharides having molecular weights ranging from about 6,000 Daltons to about 12,000 Daltons wherein at least 15% of said oligosaccharides have at least one pentasaccharide sequence that interacts with antithrombin.

36. The MMWH composition of claim 35, wherein the oligosaccharides are of sufficient length to inhibit fibrin-bound thrombin and fluid-phase thrombin by catalyzing antithrombin, and to inhibit thrombin generation by catalyzing factor Xa inactivation by antithrombin.

37. The MMWH composition of claim 35, wherein said oligosaccharides are of sufficient length to bridge antithrombin or heparin cofactor II (HCII) to thrombin but do not bridge thrombin to fibrin.

38. The MMWH composition of claim 35, wherein at least 31% of said oligosaccharides have a molecular weight greater than or equal to about 7,800.

39. The MMWH composition of claim 35, wherein said oligosaccharides have molecular weights ranging from about 8,000 Daltons to about 10,000 Daltons.

40. The MMWH composition of claim 38, wherein said oligosaccharides have molecular weights of about 8,500 Daltons.

41. The MMWH composition of claim 35, wherein at least 20% of said oligosaccharides have at least one pentasaccharide sequence.

42. The MMWH composition of claim 41, wherein at least 30% of said oligosaccharides have at least one pentasaccharide sequence.

43. The MMWH composition of claim 42, wherein at least 35% of said oligosaccharides have at least one pentasaccharide sequence.

44. The MMWH composition of claim 43, wherein at least 40% of said oligosaccharides have at least one pentasaccharide sequence.

45. The MMWH composition of claim 35, wherein said MMWH composition has an anti-factor IIa activity of about 40 U/mg to about 100 U/mg, and an anti-factor Xa activity of about 90 U/mg to about 150 U/mg.

46. The MMWH composition of claim 45, wherein said MMWH composition has an anti-factor IIa activity of about 60 U/mg to about 75 U/mg, and an anti-factor Xa activity of about 100 U/mg to about 125 U/mg.

47. The MMWH composition of claim 35, wherein said oligosaccharides have a polydispersity of 1.1 to 1.5.

48. The MMWH composition of claim 47, wherein said oligosaccharides have a polydisperisty of 1.2 to 1.4.

49. The MMWH composition of claim 35, wherein said MMWH composition has anti-factor Xa activity and anti-factor IIa activity and wherein the ratio of anti-factor Xa activity to anti-factor IIa activity is from about 2:1 to about 1:1.

50. The MMWH composition of claim 35 comprising a mixture of oligosaccharides derived from heparin having antithrombin and HCII related anticoagulant activity; having sufficient length to bridge antithrombin or HCII to thrombin, but do not bridge thrombin to fibrin; having at least 20%, 25%, 30%, 35%, or 40% oligosaccharides with at least one or more pentasaccharide sequence; and being enriched for oligosaccharides having a molecular weight range from about 8,000 to 10,000 Daltons.

51. The MMWH composition of claim 35 comprising a mixture of oligosaccharides derived from heparin having sufficient length to bridge antithrombin or HCII to thrombin, but do not bridge thrombin to fibrin; having at least 20%, 25%, 30%, 35%, or 40% oligosaccharides with at least one or more pentasaccharide sequence; having a mean molecular weight of about 8,000 to 9,8000 Daltons; and having a polydispersity of about 1.1 to about 1.5.

52. The MMWH composition of claim 35 comprising a mixture of oligosaccharides derived from heparin having sufficient length to bridge antithrombin or HCII to thrombin, but do not bridge thrombin to fibrin; having at least 20%, 25%, 30%, 35%, or 40% oligosaccharides with at least one or more pentasaccharide sequence; being enriched for oligosaccharides having a molecular weight range from about 8,000 to 10,000 Daltons; having a mean molecular weight of about 8,000 to 9,800 Daltons; and having similar anti-factor Xa and anti-factor IIa activities wherein the ratio of anti-factor Xa activity to anti-factor IIa activity is from about 2:1 to about 1:1.

53. The MMWH composition of claim 50 comprising a mixture of oligosaccharides derived from heparin having a polydispersity of about 1.1 to about 1.5.

54. A MMWH composition of claim 35 comprising a mixture of oligosaccharides derived from heparin having sufficient length to bridge antithrombin or HCII to thrombin, but do not bridge thrombin to fibrin; having a mean molecular weight of about 8,000 to 9,800 Daltons; having a polydisperisty of about 1.1 to about 1.5; having an anti-factor Xa activity from about 80 IU/mg to about 105 IU/m

55. The MMWH composition of claim 54 comprising a mixture of oligosaccharides derived from heparin wherein at least 30%, 35%, 40%, 45%, or 50% of said oligosaccharides have a molecular weight greater than or equal to 8,000 Daltons.

56. The MMWH composition of claim 53 comprising a mixture of oligosaccharides derived from heparin wherein at least 30%, 35%, 40%, 45%, or 50% of said oligosaccharides have a molecular weight greater than or equal to 8,000 Daltons: having similar anti-factor Xa and anti-factor IIa activities wherein the ratio of anti-factor Xa activity to anti-factor IIa activity is from about 2:1 to about 1:1 having an anti-factor Xa activity from about 80 IU/mg to about 105 IU/mg; and having an anti-factor IIa activity from about 20 IU/mg to about 150 IU/mg.

57. A method of treating a thrombotic condition in a subject comprising administering to said subject a pharmacologically acceptable dose of the MMWH composition of claim 35.

58. The method of claim 57, wherein said thrombotic condition is arterial thrombosis, coronary artery thrombosis, venous thrombosis, or pulmonary embolism.

59. The method of claim 57, wherein said MMWH composition is administered by injection.

60. A method of preventing the formation of a thrombus in a subject at risk of developing thrombosis comprising administering to said subject a pharmacologically acceptable dose of the MMWH composition of claim 35.

61. The method of claim 60, wherein said subject is at increased risk of developing thrombosis due to a medical condition which disrupts hemostasis.

62. The method of claim 61, wherein the medical condition is coronary artery disease, or atherosclerosis.

63. The method of claim 60, wherein said subject is at increased risk of developing thrombosis due to a medical procedure.

64. The method of claim 63, wherein the medical procedure is cardiac surgery, cardipulmonary bypass, catheterization, or atherectomy.

65. The method of claim 64, wherein the catheterization is cardiac catherterization.

66. A method of inhibiting thrombus formation in a patient comprising the step of administering to the patient a pharmacologically acceptable dose of the MMWH composition of claim 35.

67. A composition comprising the MMWH composition of claim 35 and a pharmaceutically acceptable carrier.

68. A method of treating deep vein thrombosis in a patient comprising administering to said patient undergoing orthopedic surgery a therapeutically effective amount of the MMWH composition of claim 35.

69. A method of preventing a pulmonary embolism in a subject comprising administering to said subject a therapeutically effective amount of the MMWH composition of claim 35.

70. A medium molecular weight heparin (MMWH) composition comprising a mixture enriched for sulfated oligosaccharides derived from heparin by enzymatic cleavage with heparinase having molecular weights ranging from about 8,000 Daltons to about 12,000 Daltons wherein at least 15% of said oligosaccharides have at least one pentasaccharide sequence.

71. A MMWH composition of claim 70 wherein the oligosaccharides have (a) antithrombin and HCII related anticoagulant activity; (b) sufficient length to bridge antithrombin or HCII to thrombin, but not bridge thrombin to fibrin; (c) at least 20%, 25%, 30%, 35%, or 40% oligosaccharides with at least one or more pentasaccharide sequence; and (b) a molecular weight range from about 8,000 to 10,000 Daltons.

72. A MMWH composition of claim 71 having a mean molecular weight of about 8,000 to 9,800 Daltons and having a polydisperisty of about 1.1 to about 1.5.

73. A MMWH composition of claim 71 wherein the oligosaccharides have similar anti-factor Xa and anti-factor IIa activities wherein the ratio of anti-factor Xa activity to anti-factor IIa activity is from about 2:1 to about 1:1.

74. A MMWH composition of claim 73 wherein the oligosaccharides have an anti-factor Xa activity from about 80 IU/mg to about 105 IU/mg, and an anti-factor IIa activity from about 20 IU/mg to about 150 IU/mg.

75. A medium molecular weight heparin (MMWH) composition comprising a mixture of sulfated oligosaccharides wherein (a) at least 31% of the oligosaccharides have molecular weights ranging from about 8,000 Daltons to about 10,000 Daltons; (b) at least 15% of said oligosaccharides have at least one pentasaccharide sequence; and (c) the oligosaccharides are of sufficient length to bridge antithrombin or heparin cofactor II (HCII) to thrombin but do not bridge thrombin to fibrin.

76. A MMWH composition of claim 75 having a polydisperisty of about 1.1 to about 1.5.

77. A MMWH composition of claim 75 wherein the oligosaccharides have similar anti-factor Xa and anti-factor IIa activities wherein the ratio of anti-factor Xa activity to anti-factor IIa activity is from about 2:1 to about 1:1.

78. A MMWH composition of claim 75 wherein the oligosaccharides have an anti-factor Xa activity from about 80 IU/mg to about 105 IU/mg, and an anti-factor IIa activity from about 20 IU/mg to about 150 IU/mg.

79. A method of treating a thrombotic condition in a subject comprising administering to said subject a pharmacologically acceptable dose of the MMWH composition of claim 70.

80. A method of treating a thrombotic condition in a subject comprising administering to said subject a pharmacologically acceptable dose of the MMWH composition of claim 75.
